(19) United States
(12) Patent Application Publication
(10) Pub. No.: US 2026/0335466 A1
(43) Pub. Date: Oct. 29, 2026

(54) CAPPED POLYNUCLEOTIDE, CAPPED MRNA AND COMPOSITION THEREOF, PHARMACEUTICAL PROTEIN, AND PREPARATION METHOD THEREFOR AND USE THEREOF, AND PHARMACEUTICAL PREPARATION

(71) Applicants: SHANGHAI HONGENE BIOTECH CORPORATION, Shanghai (CN); SHANGHAI HONGENE BIOENGINEERING CO., LTD, Shanghai (CN)

(72) Inventors: Feng YAO, Shanghai (CN); Yang YANG, Shanghai (CN); Dingyuan ZHANG, Shanghai (CN); Youchen LI, Shanghai (CN); Chen WANG, Shanghai (CN); Jiangbo CHEN, Shanghai (CN); Jiayan ZHAO, Shanghai (CN); Zhexian CHEN, Shanghai (CN); Zhihui ZOU, Shanghai (CN)

(73) Assignees: SHANGHAI HONGENE BIOTECH CORPORATION, Shanghai (CN); SHANGHAI HONGENE BIOENGINEERING CO., LTD, Shanghai (CN)

(21) Appl. No.: 19/149,081

(22) PCT Filed: May 13, 2024
(86) PCT No.: PCT/CN2024/092892

§ 371 Date: Jun. 10, 2026

(30) Foreign Application Priority Data
Mar. 13, 2023 (CN) 202310239920.8
Mar. 1, 2024 (CN) 202410235630.0

(51) Int. Cl. A61K 48/00; A61K 38/00; C07H 1/00; C07H 21/02; C12N 9/02

(52) U.S. Cl. CPC A61K 48/0066; C07H 1/00; C07H 21/02; C12N 9/0069; C12Y 113/12007; A61K 38/00

(57) ABSTRACT

Provided are a capped polynucleotide, capped mRNA and a composition thereof, a pharmaceutical protein, and a preparation method therefor and the use thereof, and a pharmaceutical preparation, which relate to the technical field of genetic engineering. According to the capped polynucleotide in the embodiments, at least one pentose and the adjacent phosphoric acid are connected via carbon-carbon double bonds, so that when the capped polynucleotide is used for preparing the capped mRNA, the prepared capped mRNA is more stable and has a better translation activity and a lower decomposition rate, and the capping rate can also be improved.

### CROSS-REFERENCE TO RELATED APPLICATIONS

The present disclosure claims priority to the Chinese Patent Application No. 202310239920.8 filed with the China Patent Office on Mar. 13, 2023 and entitled “CAPPED POLYNUCLEOTIDE, CAPPED MRNA AND COMPOSITION THEREOF, PHARMACEUTICAL PROTEIN AND PREPARATION METHOD THEREFOR AND USE THEREOF, AND PHARMACEUTICAL PREPARATION”, and the Chinese patent application No. 202410235630.0 filed with the China Patent Office on Mar. 1, 2024 and entitled “CAPPED POLYNUCLEOTIDE, CAPPED MRNA AND COMPOSITION THEREOF, PHARMACEUTICAL PROTEIN, AND PREPARATION METHOD THEREFOR AND USE THEREOF, AND PHARMACEUTICAL PREPARATION”, the contents of which are incorporated herein by reference in entirety.

### TECHNICAL FIELD

The present disclosure relates to the technical field of genetic engineering, and specifically to a capped polynucleotide, a capped mRNA and a composition thereof, a pharmaceutical protein and a preparation method therefor and use thereof, and a pharmaceutical preparation.

### BACKGROUND ART

Messenger RNA (mRNA) is a single-stranded polynucleotide chain carrying protein encoding information in genome, and can be used as a drug, a vaccine, etc. Currently, mRNA can be synthesized in vitro using an in vitro transcription (IT) method. After synthesis of the mRNA, the mRNA needs to be modified at 5′, and such modification structure is generally called as cap structure. The cap structure can prevent degradation of the mRNA at 5′ end, and can also serve to help the mRNA to pass through selective channels of nuclear membrane and enter cytoplasm, enhance translation, help complete an entire cleavage process, and so on.

Existing cap structures are generally based on N7 methylguanosine (M7G), and are linked to an RNA through a 5′,5′-triphosphate bridge (m7GpppN). A structure of such cap structure is called as Cap 0, but the Cap 0 structure does not have a high capping rate; the capped mRNA also has poor stability and a high degradation rate in organisms, and translational activity is low when the mRNA containing such cap structure is used for translation of protein.

### SUMMARY

Embodiments of the present disclosure aim at providing a capped polynucleotide, a capped mRNA and a composition thereof, a pharmaceutical protein and a preparation method therefor and use thereof, and a pharmaceutical preparation, so that stability and capping rate of the capped mRNA can be enhanced, a degradation rate of the capped mRNA in organisms can be decreased, and translational activity of the capped mRNA can be improved.

In the first aspect, embodiments of the present disclosure provide a capped polynucleotide, which is a compound of a following general structural formula, or in a salt form of the compound of the following general structural formula:

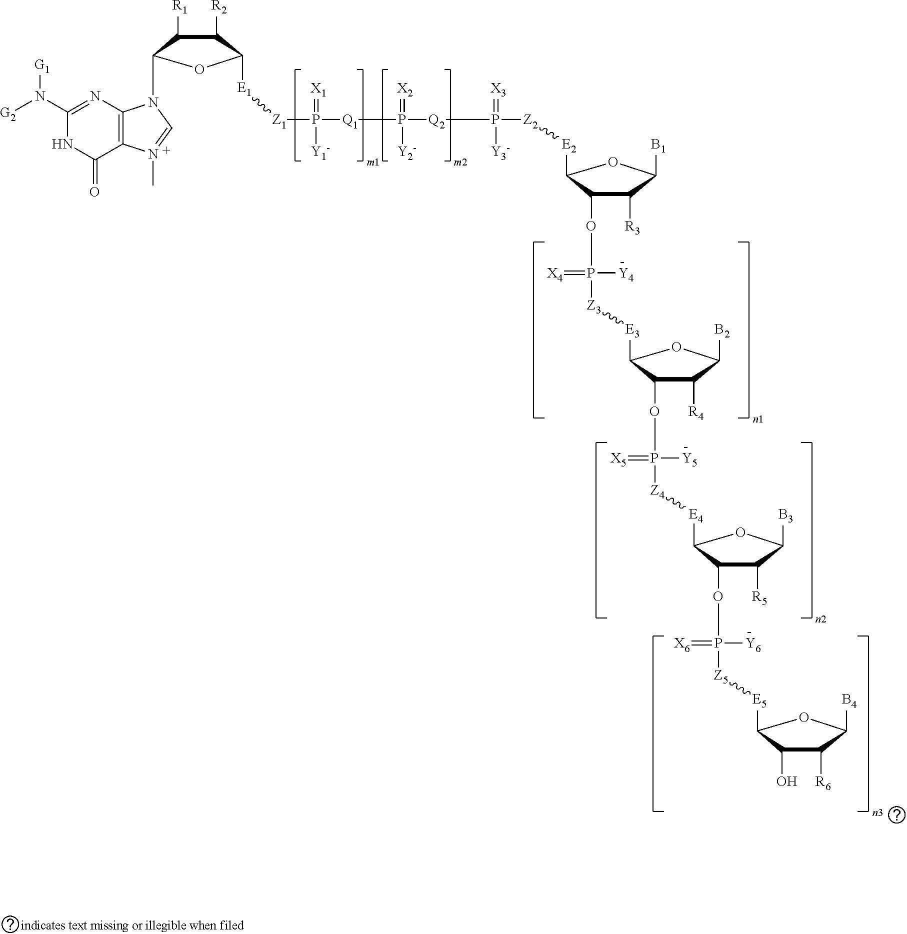

Each of E₁ to E₅ is carbon, each of Z₁ to Z₅ is independently carbon or oxygen, and at least one position between E₁ and Z₁, between E₂ and Z₂, between E₃ and Z₃, between E₄, and Z₄, and between E₅ and Z₅ is a carbon-carbon double bond; each of B₁ to B₄ is independently a nucleobase; each of m₁ and m₂ is independently 0 or 1; n₁ is 1, and each of n₂ and n₃ is independently any integer between 0 and 20; each of G₁ and G₂ is independently selected from hydrogen and methyl; each of R₁ to R₆ is independently any of hydrogen, hydroxyl, alkyl, alkoxy, and halo; each of X₁ to X₆ is independently any of oxygen, sulfur, ═NH, and ═NCH₃; each of Y₁ to Y₆ is independently any of oxygen, sulfur, selenium, aryl, alkyl, alkoxy, aryloxy, —BH₂, —NH, —NCH₃, and —N(C(═O)CH₃); and each of Q₁ and Q₂ is independently selected from the group consisting of oxygen, sulfur, and methylene.

In the above technical solution, the inventors find that since the capped polynucleotide of the above general structural formula has a carbon-carbon double bond in at least one position between E₁ and Z₁, between E₂ and Z₂, between E₃ and Z₃, between E₄ and Z₄, and between E₅ and Z₅, when the above capped polynucleotide is used to cap 5′ of mRNA fragments, stability and capping rate of the mRNA fragments can be significantly improved, the decomposition rate of the capped mRNA fragments in organisms can be reduced, and at the same time, the mRNA fragments containing the capped polynucleotide, when subsequently used for translating proteins, also has high translational activity.

In a possible implementation, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond; each of B₁ to B₄ is independently a nucleobase; each of m₁ and m₂ is independently 0 or 1; n₁ is 1, and a sum of n₁, n₂, and n₃ is not greater than 5; each of G₁ and G₂ is independently selected from hydrogen and methyl; each of R₁ to R₆ is independently any of hydrogen, hydroxyl, and alkoxy; each of X₁ to X₆ is independently any of oxygen and sulfur; each of Y₁ to Y₆ is independently any of oxygen and sulfur, and each of Q₁ and Q₂ is independently any of oxygen and sulfur.

The above technical solution is conducive to further improving the stability and capping rate of the mRNA fragments, further reducing the degradation rate of the mRNA fragments in organisms, and further improving the translational activity of the mRNA fragments containing the capped polynucleotide when subsequently used for translating proteins.

In a possible implementation, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond; each of B₁ to B₄ is independently a nucleobase; n₁ is 1, and a sum of n₁, n₂, and n₃ is not greater than 5; each of m₁ and m₂ is independently 0 or 1; each of G1 and G2 is independently selected from hydrogen and methyl; each of R₁ to R₆ is independently any of hydroxyl and alkoxy; each of X₁ to X₆ is independently any of oxygen and sulfur; each of Y₁ to Y₆ is independently any of oxygen and sulfur; and each of Q₁ and Q₂ is independently any of oxygen and sulfur.

The above technical solution is conducive to further improving the stability and capping rate of the mRNA fragments, further reducing the degradation rate of the mRNA fragments in organisms, and further improving the translational activity of the mRNA fragments containing the capped polynucleotide when subsequently used for translating proteins.

In a possible implementation, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond; each of B₁ to B₄ is independently a nucleobase; m₁ and m₂ are both 1; n₁ is 1, and a sum of n₁, n₂, and n₃ is not greater than 5; each of G1 and G2 is independently selected from hydrogen and methyl; R₁ and R₄ to R₆ are all hydroxyl, R₂ and R₃ are both alkoxy; or R₂ and R₄ to R₆ are all hydroxyl, R₁ and R₃ are both alkoxy; each of X₁ to X₆ is independently any of oxygen and sulfur; each of Y₁ to Y₆ is independently any of oxygen and sulfur; and each of Q₁ and Q₂ is independently any of oxygen and sulfur.

In the above technical solution, the inventors find that selection of R₁, R₂ and R₃ may further affect the stability, the capping rate and the translational activity of the mRNA fragments, and is conducive to further improving the stability and capping rate of the mRNA fragments, reducing the degradation rate of the mRNA fragments in organisms, and further improving the translational activity of the mRNA fragments containing the capped polynucleotide when subsequently used for translating proteins.

In a possible implementation, the capped polynucleotide is a compound of a following general structural formula, or the capped polynucleotide is in a salt form of the compound of the following general structural formula:

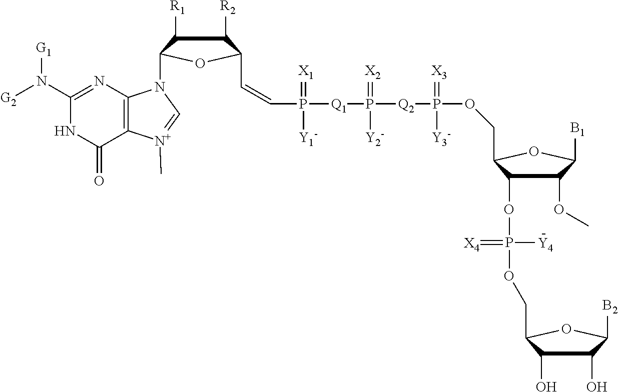

B₁ and B₂ are nucleobases respectively; each of G1 and G2 is independently selected from hydrogen and methyl; R₁ is hydroxyl, and R₂ is methoxy; or R₂ is hydroxyl, and R₁ is methoxy; each of X₁ to X₄ is independently any of oxygen and sulfur, each of Y₁ to Y₄ is independently any of oxygen and sulfur; and each of Q₁ and Q₂ is independently any of oxygen and sulfur.

In the above technical solution, when “R₁ is hydroxyl, and R₂ is methoxy; or R₂ is hydroxyl, and R₁ is methoxy”, it is conducive to further improving the stability and capping rate of the mRNA fragments, further reducing the degradation rate of the mRNA fragments in organisms, and further improving the translational activity of the mRNA fragments containing the capped polynucleotide when subsequently used for translating proteins.

In a possible implementation, the capped polynucleotide is a compound of the following general structural formula, or in a salt form of the compound of the following general structural formula:

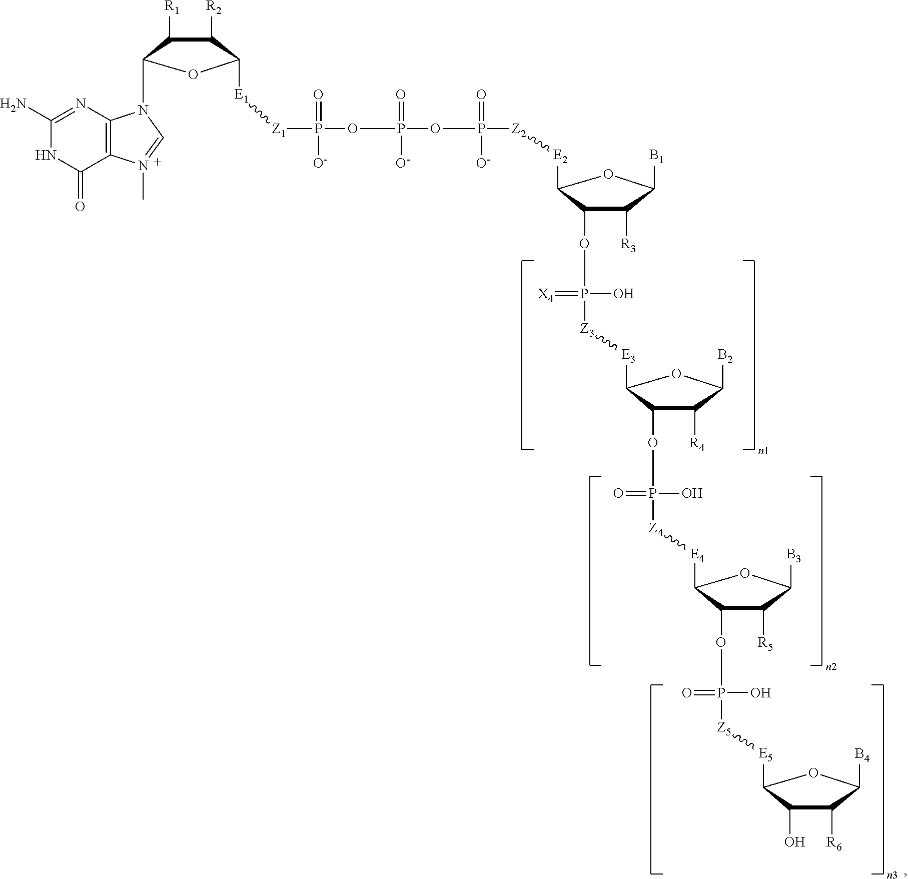

In this general structural formula, each of E₁ to E₅ is carbon, each of Z₁ to Z₅ is independently carbon or oxygen, and at least one pentose and an adjacent phosphate group are linked through carbon-carbon double bonds; each of B₁ to B₄ is independently a nucleobase; n₁ is 1, and a sum of n₁, n₂, and n₃ is not greater than 5; each of R₁ to R₆ is independently any of hydrogen, hydroxyl, and methoxy; each of G1 and G2 is independently selected from hydrogen and methyl; each of R₁ to R₆ is independently any of hydrogen, hydroxyl, and methoxy; and X₄ is oxygen or sulfur.

In a possible implementation, the carbon-carbon double bonds in the capped polynucleotide are in at least one of Z-configuration and E-configuration.

In the above technical solution, due to presence of the carbon-carbon double bonds, the capped polynucleotide is necessarily in a cis configuration (i.e. Z-configuration) and a trans configuration (i.e. E-configuration), and the inventors find that the carbon-carbon double bonds, no matter being in the Z-configuration or the E-configuration, can significantly improve the stability and capping rate of the mRNA fragments, and reduce the degradation rate of the mRNA fragments in organisms.

In a possible implementation, the capped polynucleotide is any of following compounds or in a salt form thereof.

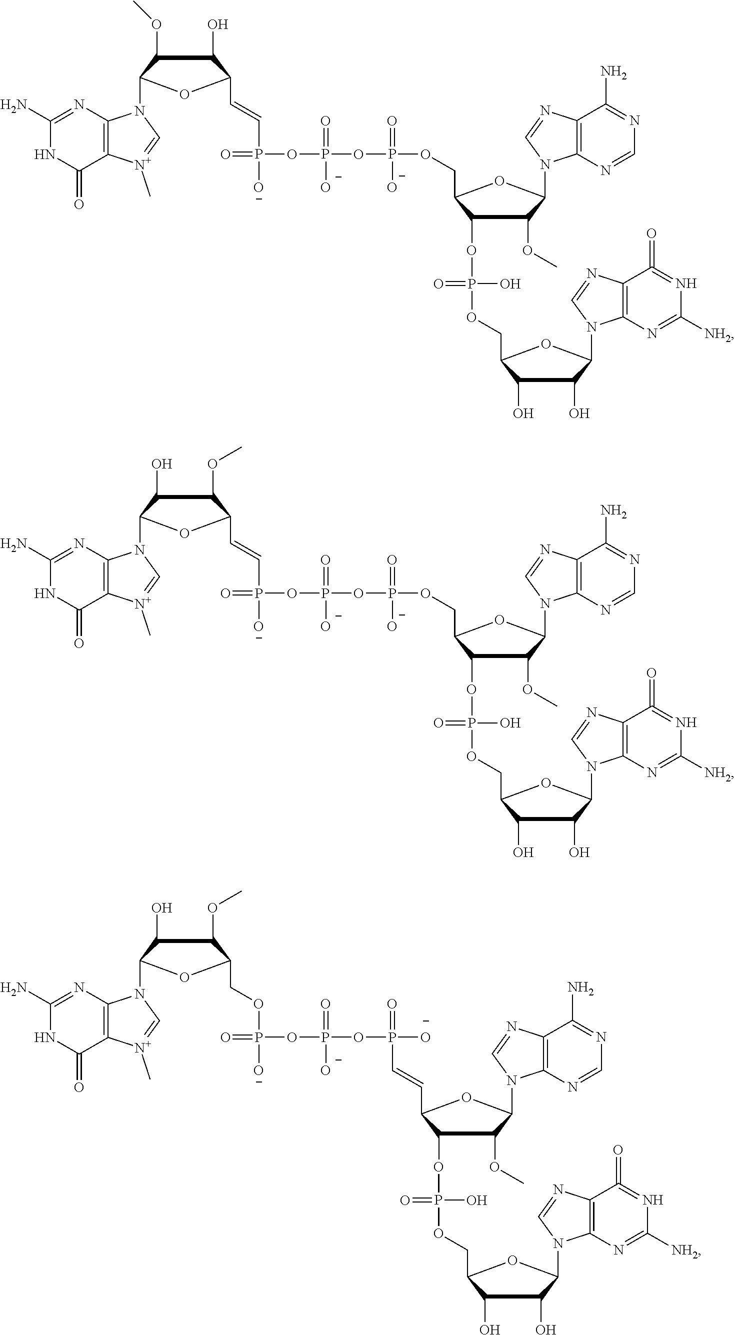

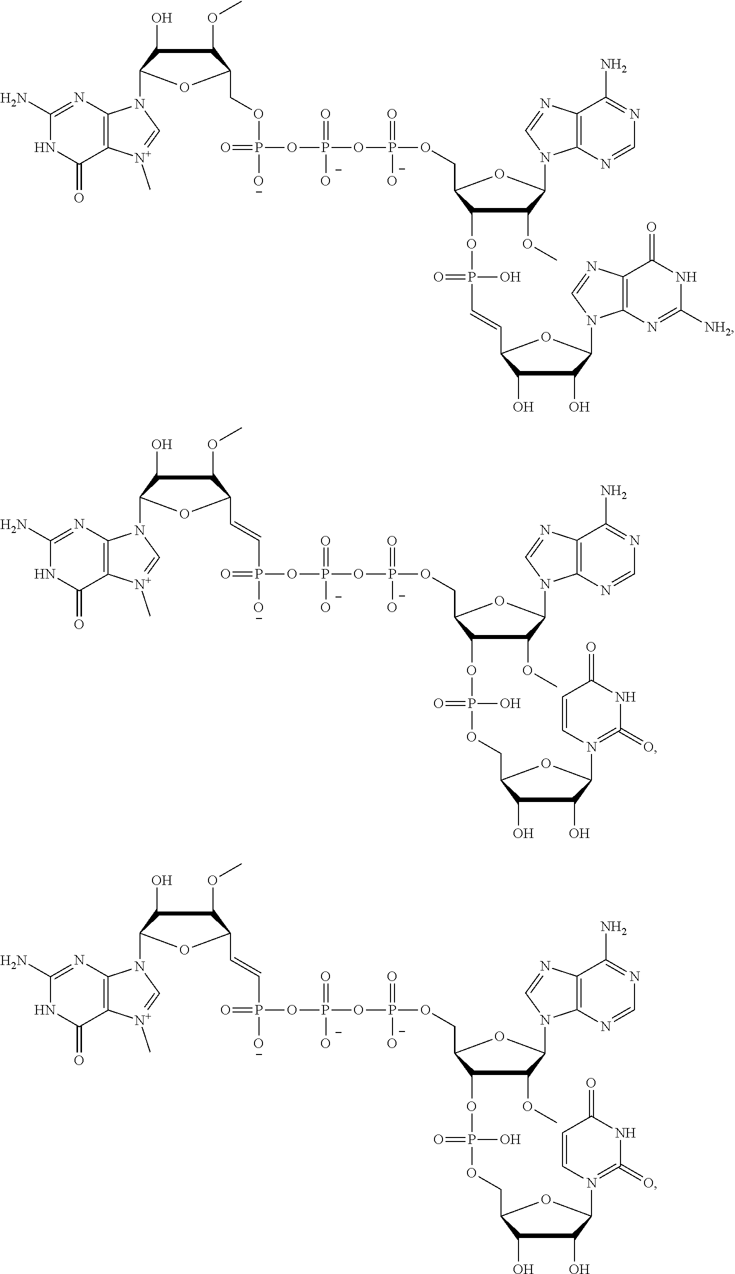

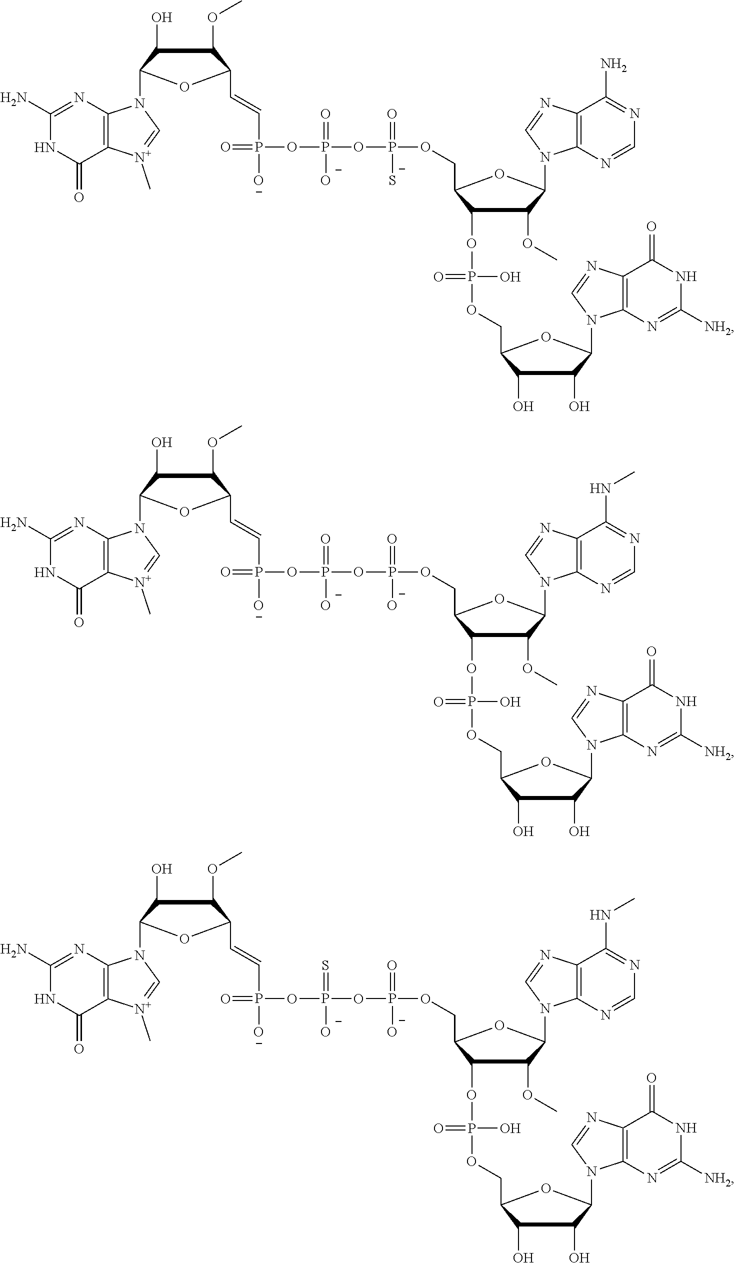

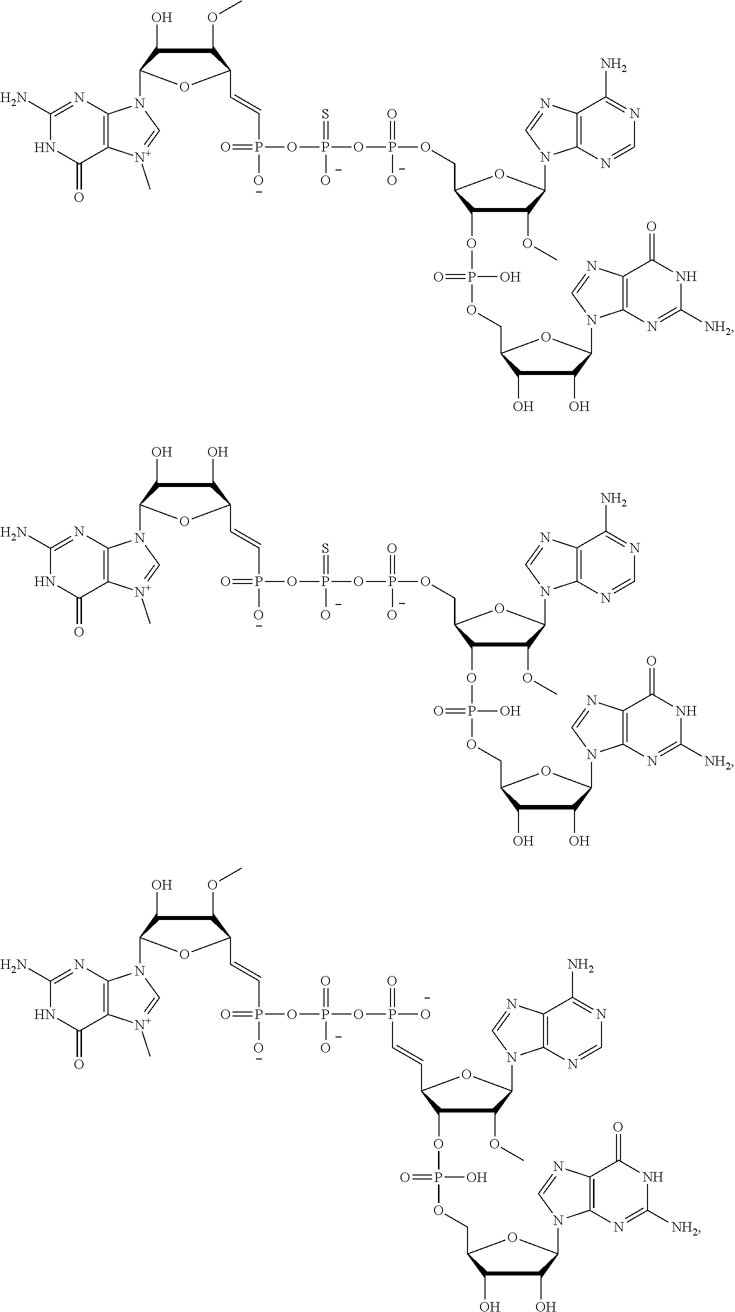

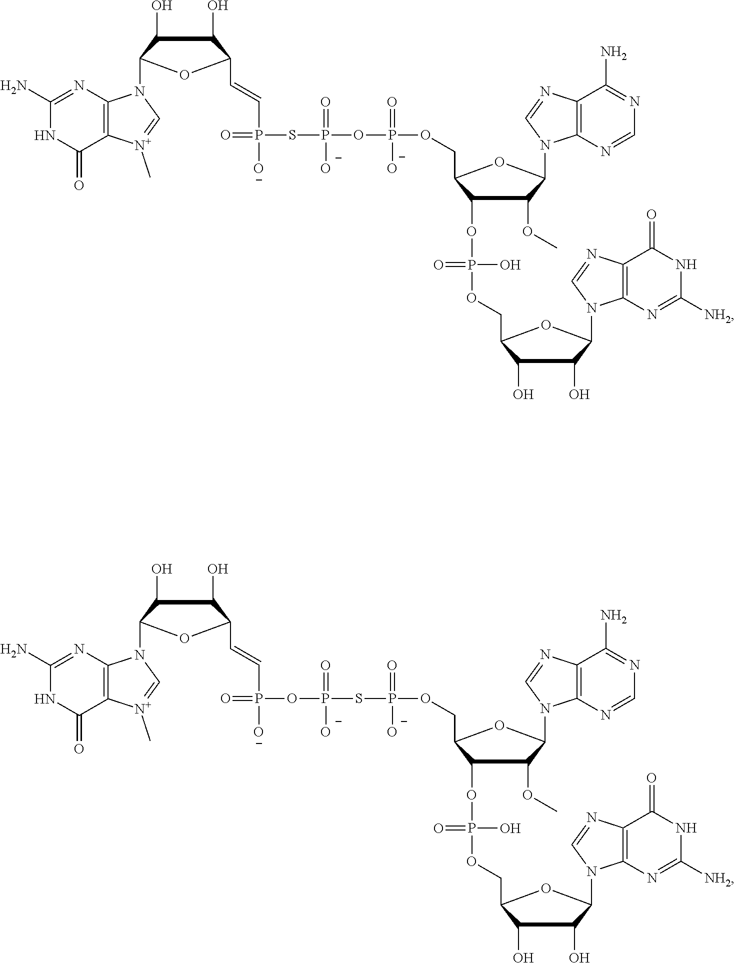

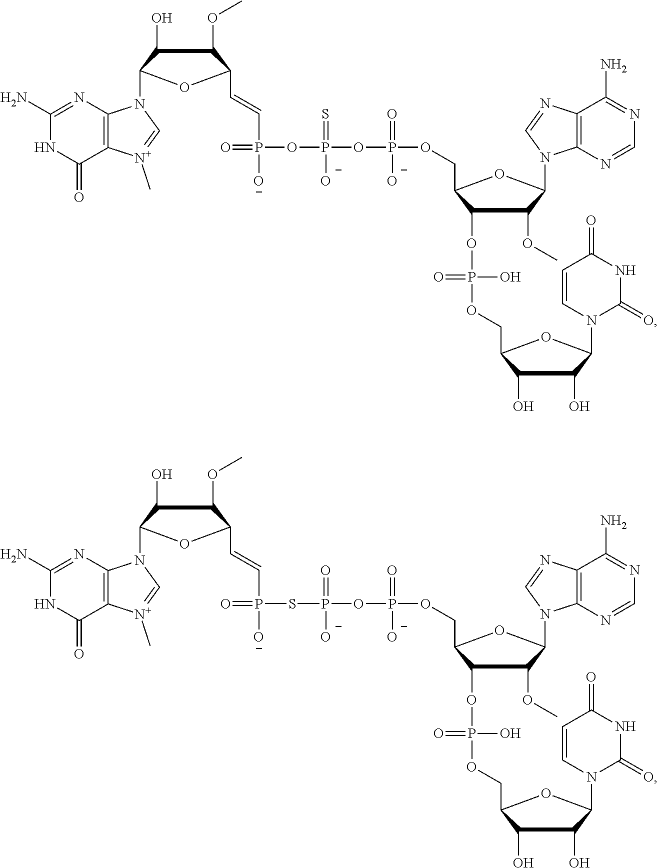

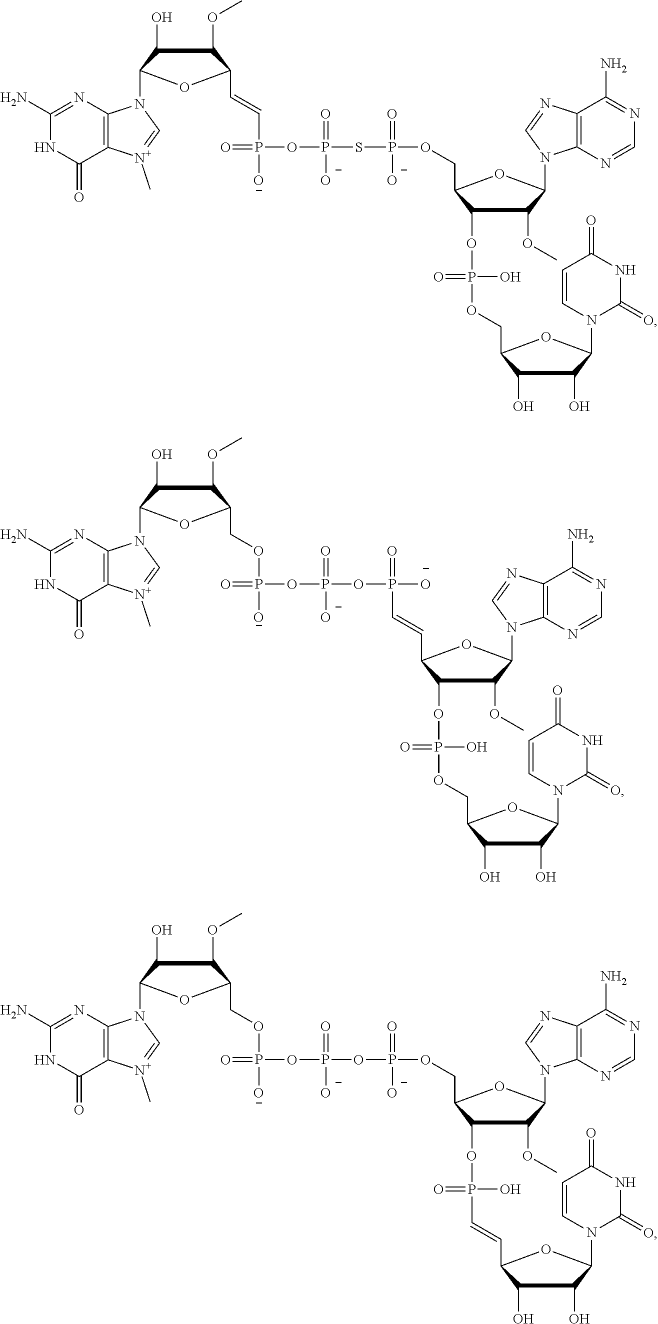

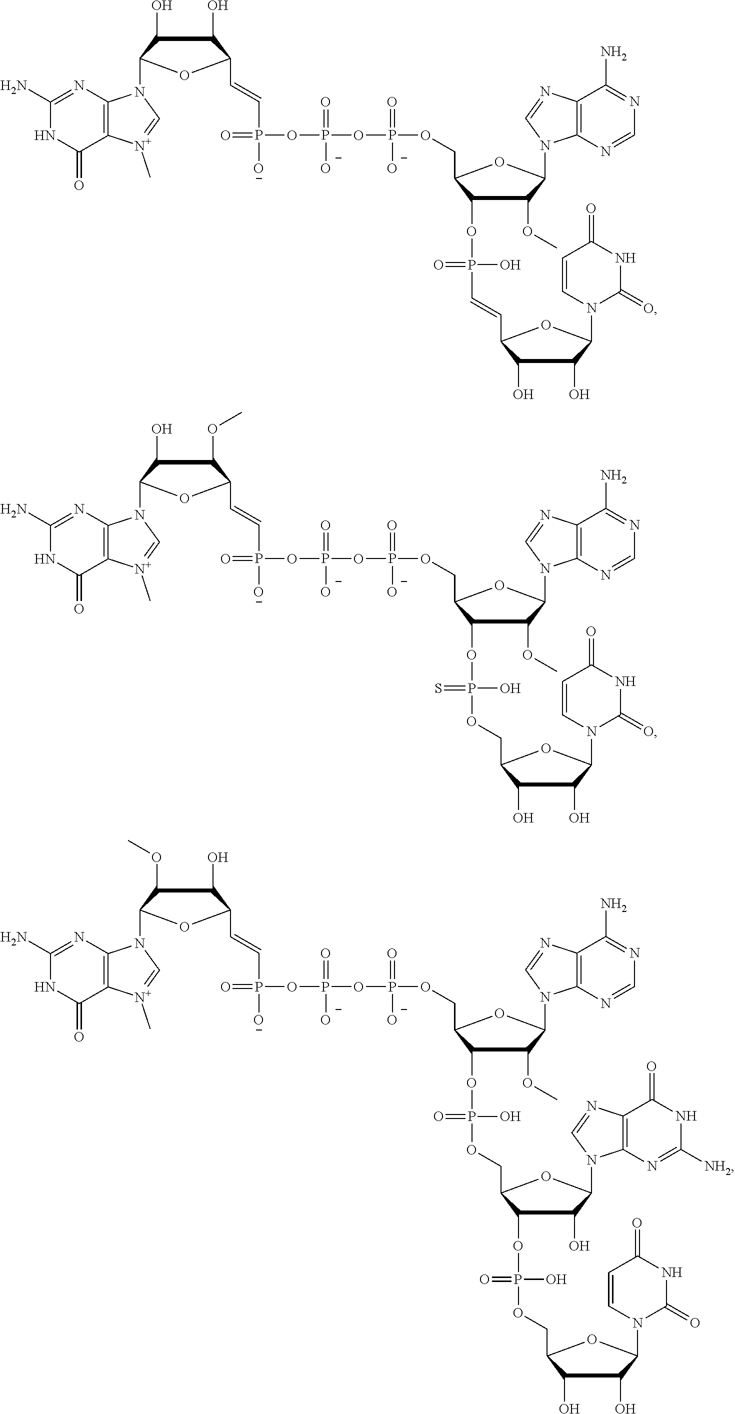

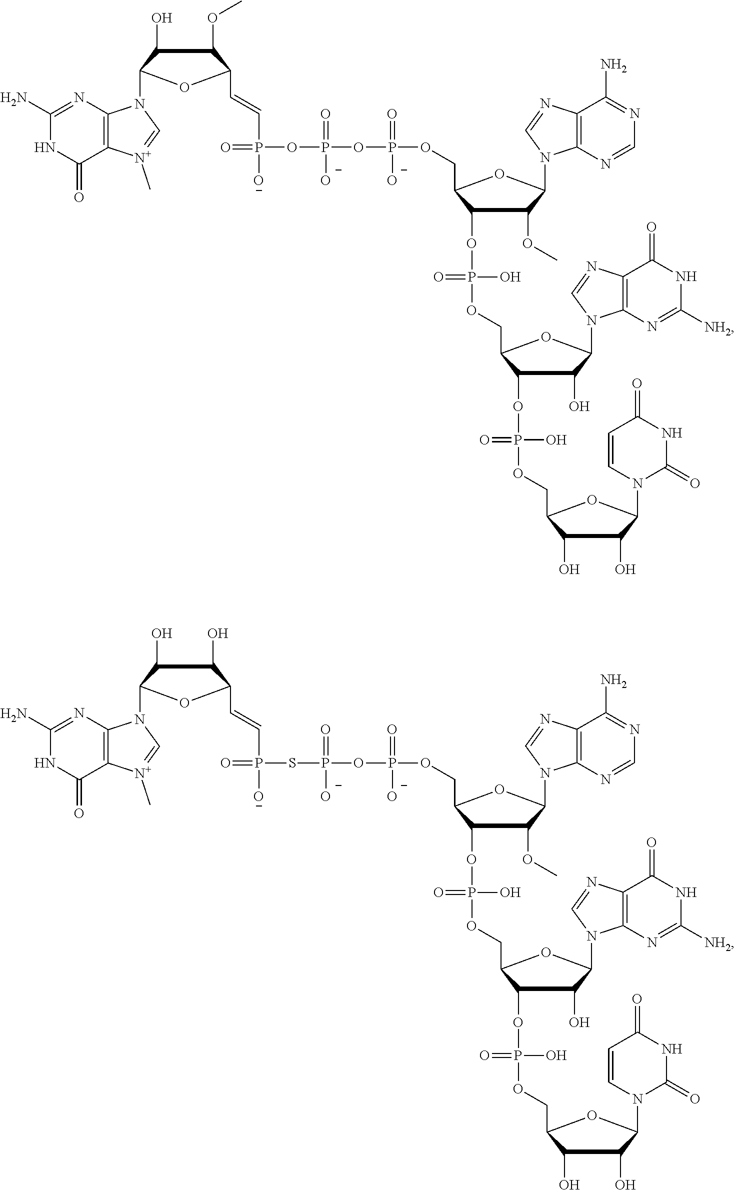

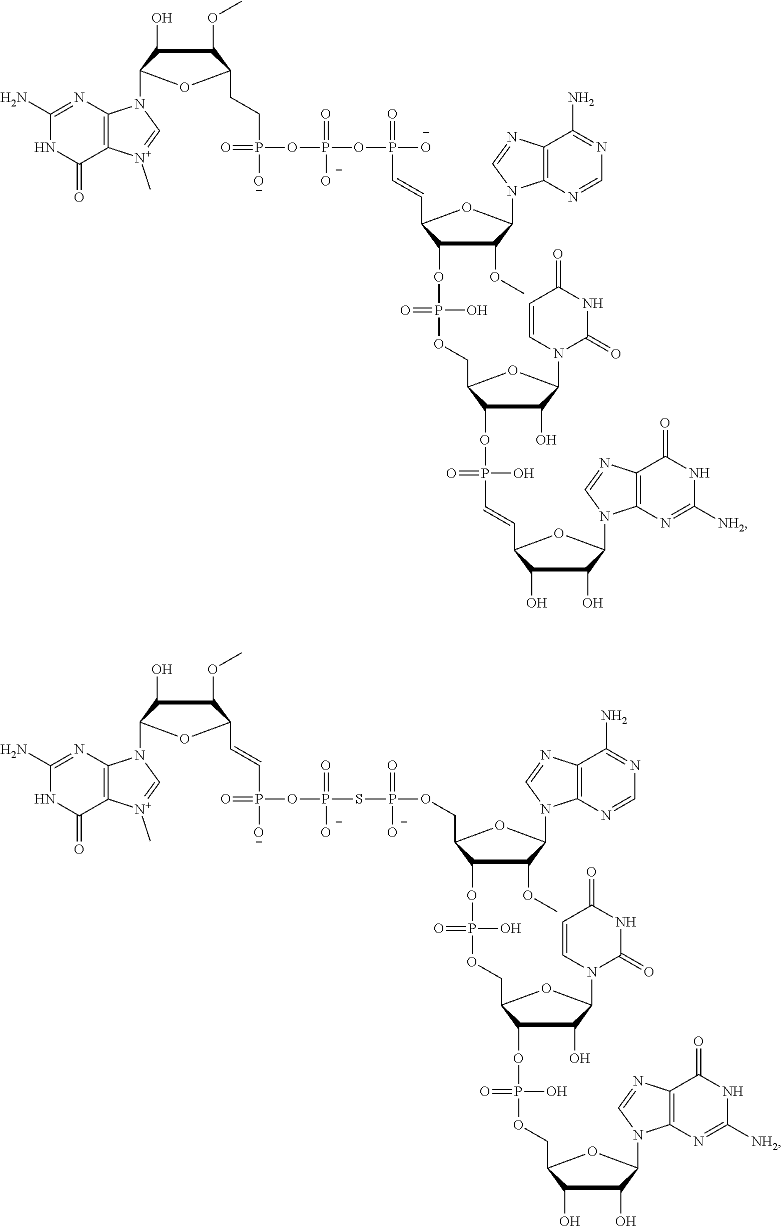

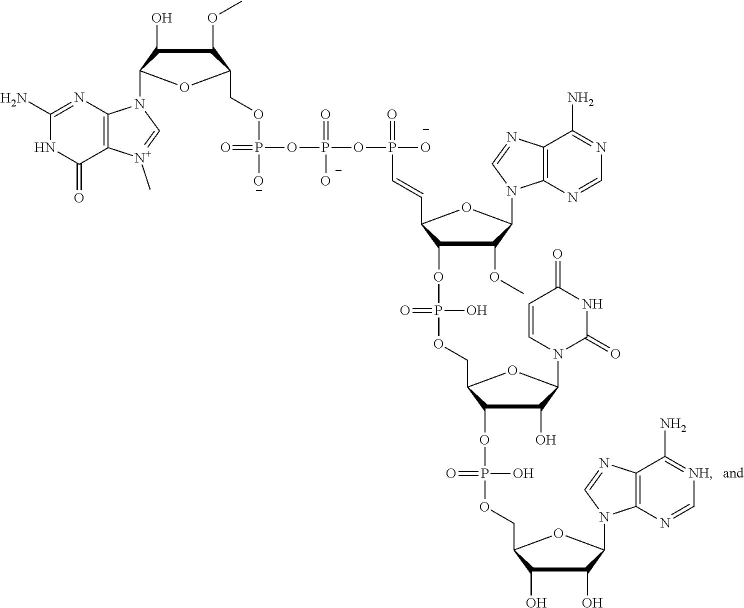

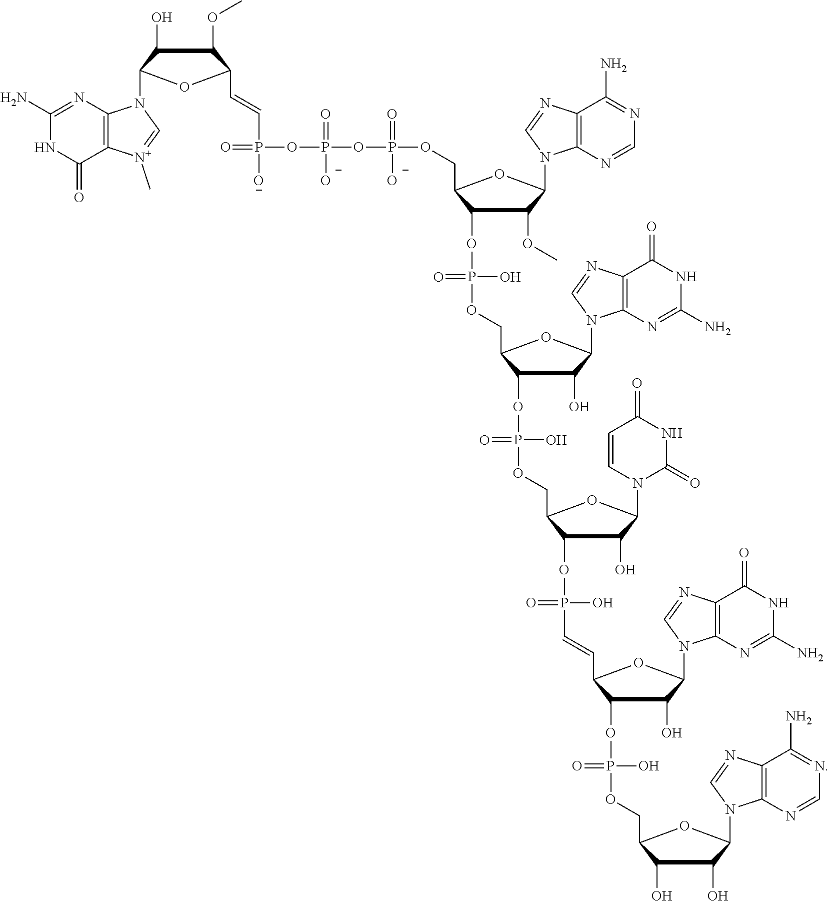

In the above technical solution, the cap structure can better improve performance of the mRNA fragments.

In a possible implementation, the capped polynucleotide is any of following compounds or in a salt form thereof:

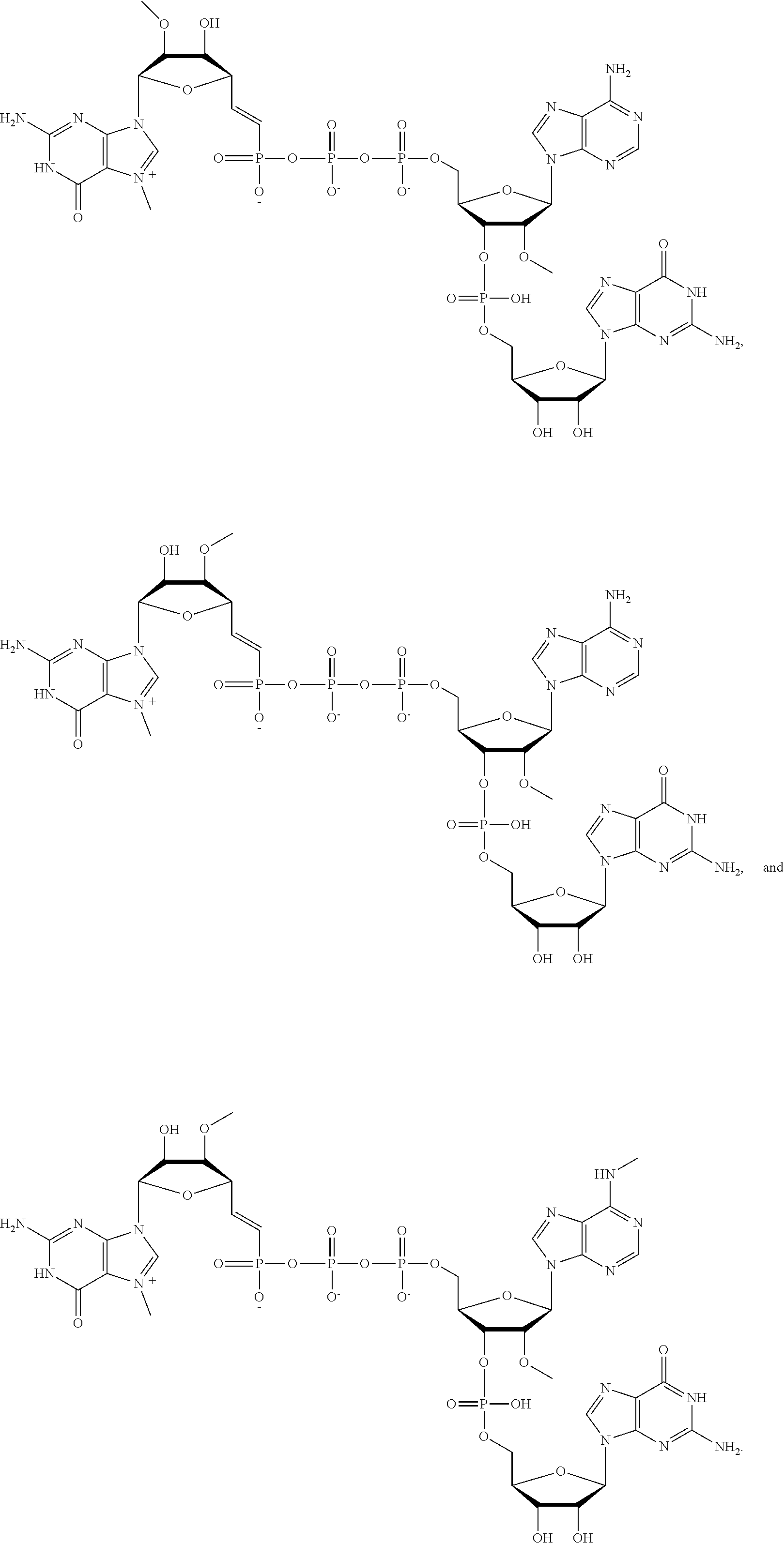

The above technical solution is conducive to further improving the stability and capping rate of the mRNA fragments, further reducing the degradation rate of the mRNA fragments in organisms, and further improving the translational activity of the mRNA fragments containing the capped polynucleotide when subsequently used for translating proteins.

In a possible implementation, the nucleobase include any of natural nucleobase or modified nucleobase. Optionally, the natural nucleobase is any one selected from adenine, uracil, guanine, hypoxanthine, cytosine, thymine, adenine derivative, uracil derivative, guanine derivative, cytosine derivative, and thymine derivative; and optionally, in the modified nucleobase, a modifying group is any one selected from N⁶-methyladenine, N¹-methyladenine, N⁶-2′-O-dimethyladenosine, pseudouridine, N¹-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N¹-methylcytosine, 5-hydroxymethylcytosine, hypoxanthine, N¹-methylguanine, N¹-methylguanine, and isoguanine.

In the second aspect, embodiments of the present disclosure provide a composition for preparing a capped mRNA, where components thereof include a template DNA and the above capped polynucleotide, and the template DNA is designed to contain a transcription site complementary to the capped polynucleotide.

In the above technical solution, the above composition can be prepared into the capped mRNA with high stability, low decomposition rate, and good translational activity.

In the third aspect, embodiments of the present disclosure provide a preparation method for a capped mRNA, including a step of performing transcription using the composition for preparing the capped mRNA in the second aspect.

In the above technical solution, the capping rate of the capped mRNA can be improved by the above preparation method.

In the fourth aspect, embodiments of the present disclosure provide a capped mRNA, including a main body mRNA fragment and the above capped polynucleotide, 5′ of the main body mRNA fragment being linked with the capped polynucleotide.

In the above technical solution, the capped mRNA has better stability, is less prone to degradation in organisms, and at the same time, can also translate proteins more efficiently.

In the fifth aspect, embodiments of the present disclosure provide a preparation method for a pharmaceutical protein, including a step of performing translation with the capped mRNA in the fourth aspect as a template.

In the above technical solution, the above preparation method for a pharmaceutical protein has higher efficiency.

In the sixth aspect, embodiments of the present disclosure provide a pharmaceutical protein, prepared by the preparation method for a pharmaceutical protein in the fifth aspect.

In the seventh aspect, embodiments of the present disclosure provide use of the capped mRNA in preparation of a medicament or a vaccine for treatment of cancer.

In the above technical solution, the above mRNA has good stability and is less prone to degradation, and it can impart good therapeutic and preventive effects to the medicament or vaccine when used for preparation of a medicament and a vaccine.

In the eighth aspect, embodiments of the present disclosure provide a pharmaceutical preparation, including the above capped mRNA, and a vector for carrying the capped mRNA, wherein the vector includes any of a lipid nanoparticle, a liposome, a polymer nanoparticle, a solid lipid nanoparticle, and an emulsion.

### BRIEF DESCRIPTION OF DRAWINGS

FIG. 1 is a schematic diagram of effects of different capped polynucleotides on cell viability;

FIG. 2 is a schematic diagram of effects of different capped polynucleotides on GFP positive rate;

FIG. 3 is a schematic diagram of effects of different capped polynucleotides on GFP MFI;

FIG. 4 is a schematic diagram of effects of different capped polynucleotides on total GFP expression amount;

FIG. 5 is an in vivo imaging diagram of mice:

FIG. 6 is an imaging diagram of organs of sacrificed mice;

FIG. 7 is a statistic chart of Flux values of organs of mice injected with Fluc LNP (at a dose of 1 μg) and then sacrificed; and

FIG. 8 is a statistic chart of Flux values of organs of mice injected with Fluc LNP (at a dose of 10 μg) and then sacrificed.

### DETAILED DESCRIPTION OF EMBODIMENTS

Embodiments of the present disclosure will be described in detail below in combination with examples, while those skilled in the art would understand that the following examples are merely used for illustrating the present disclosure, but should not be considered as limitation to the scope of the present disclosure. Examples, for which no concrete conditions are specified, are carried out according to conventional conditions or conditions recommended by manufactures. If manufacturers of reagents or apparatuses used are not specified, all of them are conventional products commercially available.

“Alkyl” refers to an aliphatic hydrocarbon C—H group. An alkyl portion may be a saturated alkyl group (referring to not containing any unsaturated unit, such as carbon-carbon double bond or carbon-carbon triple bond) or the alkyl portion may be an unsaturated alkyl group (referring to containing at least one unsaturated unit). The alkyl portion, whether saturated or unsaturated, may be branched or straight chain. There may be 1 to 8 carbon atoms (note that whenever a numerical range like “1 to 8” is mentioned herein, it refers to each integer in the given range, for example, “1 to 8 carbon atoms” means that the alkyl group may contain 1 carbon atom, 2 carbon atoms, 3 carbon atoms, etc., up to 8 carbon atoms, though the present definition also includes presence of the term “alkyl” when no numerical range is specified). Typical alkyl groups include, but are not limited to, methyl, ethyl, propyl, isopropyl, butyl, isobutyl, tert-butyl, pentyl, hexyl, and the like. “Halo” refers to fluorine, chlorine, bromine, and iodine.

In embodiments of the present disclosure, a consensus sequence of the most active class III T7 promoter includes a 17 bp sequence upstream of a transcription start site and a 6 bp sequence downstream (Cell 16: 815-25. (1979)). A position of the first transcribed nucleotide is typically referred to as +1 transcript nucleotide of RNA, the second transcribed nucleotide as +2 transcript nucleotide, and so on.

A capped polynucleotide, a composition for preparing the capped mRNA, a capped mRNA, a pharmaceutical protein and a preparation method therefor and use thereof, and a pharmaceutical preparation in the embodiments of the present disclosure will be specifically described as follows.

In the embodiments of the present disclosure, the capped polynucleotide is a compound of a following general structural formula, or in a biologically acceptable salt form of the compound of the following general structural formula:

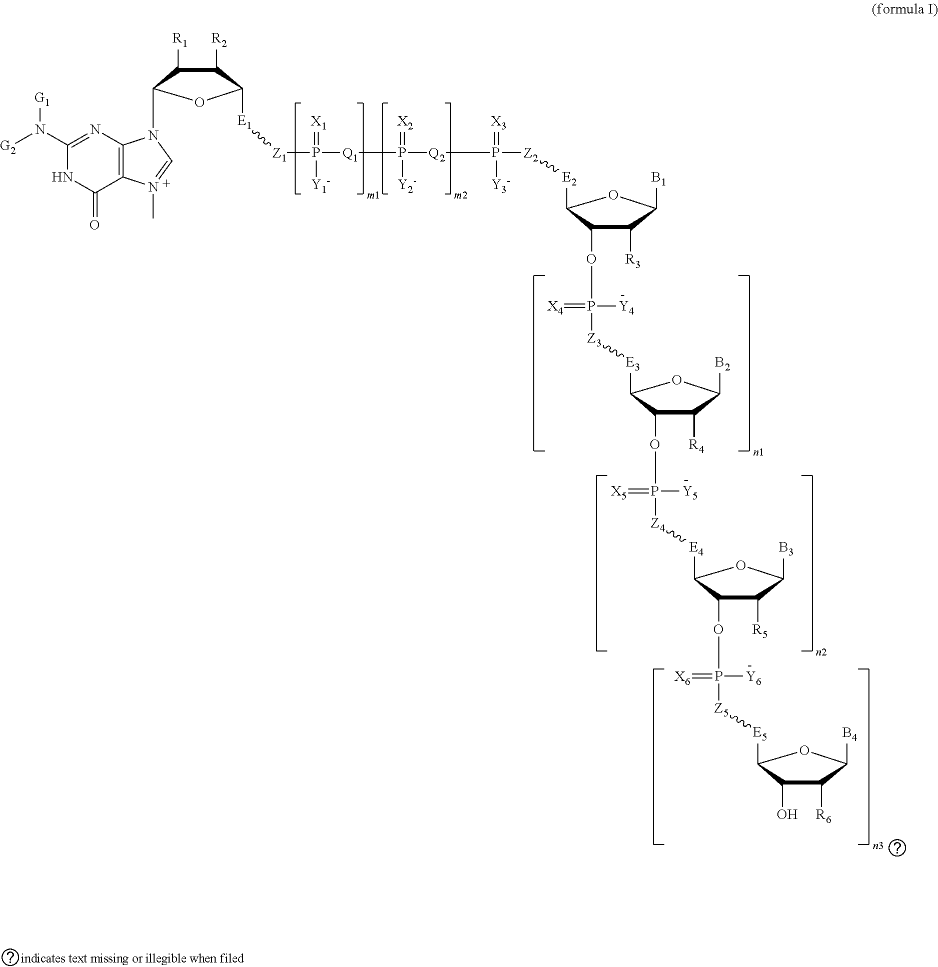

where in this general structural formula, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond. Due to the presence of such structure of the carbon-carbon double bond, the capped polynucleotide, when used at 5′ of an mRNA fragment, can significantly improve stability of the mRNA fragment, reduce decomposition rate of the mRNA fragment, and improve translational activity of the mRNA fragment.

In addition, in the above general structural formula, each of B₁ to B₄ is independently a nucleobase; each of m₁ and m₂ is independently 0 or 1; n₁ is 1, and each of n₂ and n₃ is independently any integer between 0 and 20; each of G1 and G2 is independently selected from hydrogen and methyl; each of R₁ to R₆ is independently any of hydrogen, hydroxyl, alkyl (such as long-chain alkyl), alkoxy, and halo, or a group having electric, magnetic, and optical signals, etc.; each of X₁ to X₆ is independently any of oxygen, sulfur, ═NH, and ═NCH₃; each of Y₁ to Y₆ is independently any of oxygen, sulfur, selenium, aryl, alkyl, alkoxy, aryloxy, —BH₂, —NH, —NCH₃, and —N(C(═O)CH₃); and each of Q₁ and Q₂ is independently selected from the group consisting of oxygen, sulfur, and methylene.

In the above, the “nucleobase” in the embodiments of the present disclosure may be natural nucleobase or modified nucleobase. The natural nucleobase is generally any one selected from adenine (A), guanine (G), cytosine (C), uracil (U), thymine (T) and derivatives thereof: the modified nucleobase refers to a compound obtained by substituting one or two or more hydrogens of a natural base with a modifying group, such as, N⁶-methyladenine. In the present embodiment, the modifying group is any one selected from N⁶-methyladenine, N¹-methyladenine, N⁶-2′-O-dimethyladenosine, pseudouridine, N¹-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N¹-methylcytosine, 5-hydroxymethylcytosine, hypoxanthine, N¹-methylguanine, N¹-methylguanine, and isoguanine.

It could be understood that, in the present disclosure, Y₁ to Y₆ in the structural formula of the capped polynucleotide in the present disclosure may be negatively charged or uncharged. In the above, when Y₁ to Y₆ in the structural formula of the capped polynucleotide are uncharged, the structural formula of the capped polynucleotide may be as follows:

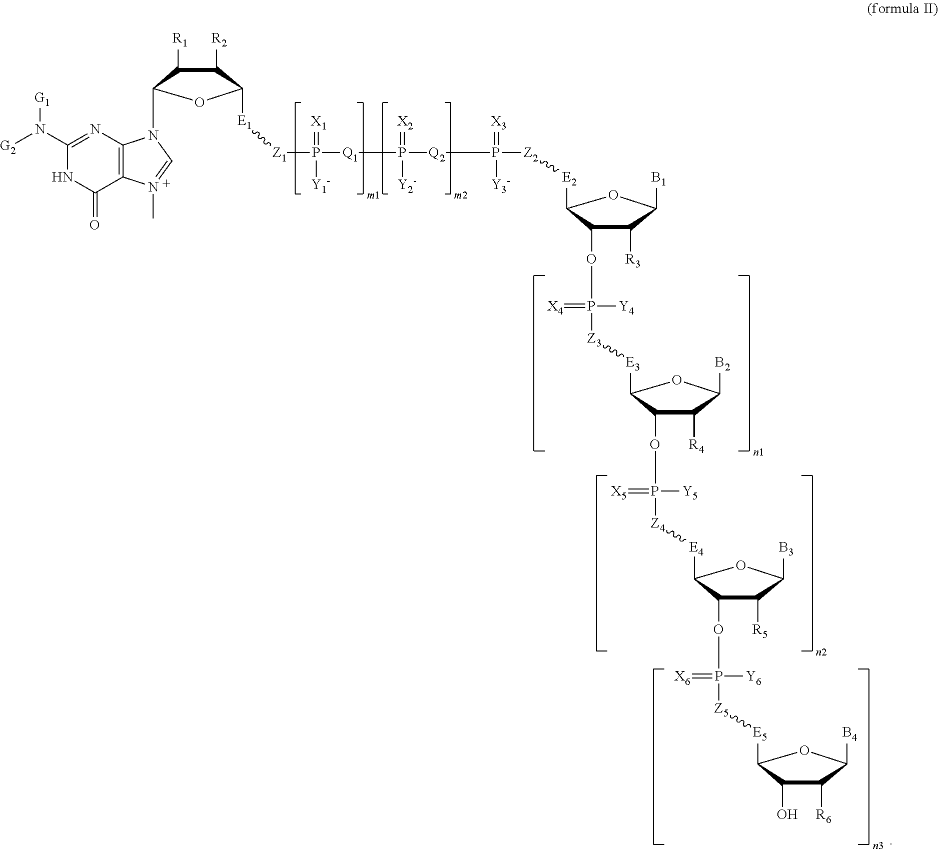

In formula II, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond; each of B₁ to B₄ is independently a nucleobase; each of m₁ and m₂ is independently 0 or 1; n₁ is 1, and each of n₂ and n₃ is independently any integer between 0 and 20; each of G₁ and G₂ is independently selected from hydrogen and methyl; each of R₁ to R₆ is independently any of hydrogen, hydroxyl, alkyl, alkoxy, and halo; each of X₁ to X₆ is independently any of oxygen, sulfur, ═NH, and ═NCH₃; each of Y₁ to Y₆ is independently any of hydroxy, mercapto, selenium, aryl, alkyl, alkoxy, aryloxy, —BH₃, —NH₂, —NHCH₃, and —NH(C(═O)CH₃); and each of Q₁ and Q₂ is independently selected from the group consisting of oxygen, sulfur, and methylene.

When one or more of Y₁ to Y₆ in the structural formula II of the capped polynucleotide are negatively charged, that is, “at least one of Y₁ to Y₆” in formula II loses proton H, for example, “Y₁” in formula II may be “Y₁⁻”, “Y₂” in formula II may be “Y₂⁻”, “Y₃” in formula II may be “Y₃⁻”, “Y₄” in formula II may be “Y₄⁻”, “Y₅” in formula II may be “Y₅⁻”, and “Y₆” in formula II may be “Y₆⁻”. Exemplarily, when “Y₁” is “Y₁⁻”, “Y₁⁻” may be any of oxygen, sulfur, selenium, aryl, alkyl, alkoxy, aryloxy, —BH₂, —NH, —NCH₃, —N(C(═O)CH₃), and when “Y₂ is Y₂⁻”, “Y₃ is Y₃⁻”, “Y₄ is Y₄⁻”, “Y₅ is Y₅⁻” and/or “Y₆ is Y₆⁻”, each of Y₂⁻ to Y₆⁻ is independently any of oxygen, sulfur, selenium, aryl, alkyl, alkoxy, aryloxy, —BH₂, —NH, —NCH₃, and —N(C(═O)CH₃).

It should be noted that formula III

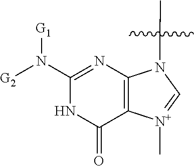

in formula I or formula II also exists in the form of

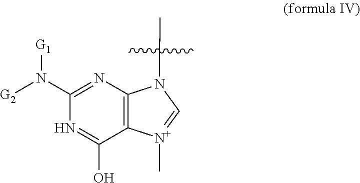

in the capped polynucleotide; and further, formula IV

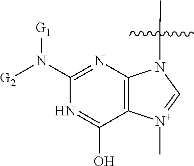

also exists in the form of

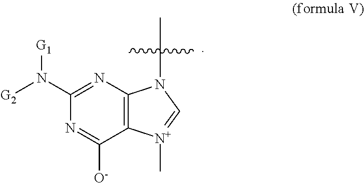

In some optional embodiments of the present disclosure, in formula I, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond; each of B₁ to B₄ is independently a nucleobase; each of m1 and m2 is independently 0 or 1; n₁ is 1, and a sum of n₁, n₂, and n₃ is not greater than 5; each of G1 and G2 is independently selected from hydrogen and methyl; each of R₁ to R₆ is independently any of hydrogen, hydroxyl, and alkoxy; each of X₁ to X₆ is independently any of oxygen and sulfur; each of Y₁ to Y₆ is independently any of oxygen and sulfur; and each of Q1 and Q2 is independently any of oxygen and sulfur.

The above method is conducive to further improving stability and capping rate of mRNA fragments, further reducing the degradation rate of mRNA fragments in organisms, and further improving translational activity of the mRNA fragments containing the capped polynucleotide when subsequently used for translating proteins.

Further, in formula I, each of E₁ to E₅ is carbon, each of Z₁ to Z₅ is independently carbon or oxygen, and at least one position between E₅ and Z₅ between E₂ and Z₂, between E₃ and Z₃, between E₄ and Z₄, and between E₅ and Z₅ is a carbon-carbon double bond; each of B₁ to B₄ is independently a nucleobase; n₁ is 1, and a sum of n₁, n₂, and n₃ is not greater than 5; each of m1 and m2 is independently 0 or 1; each of G1 and G2 is independently selected from hydrogen and methyl; each of R₁ to R₆ is independently any of hydroxyl and alkoxy; each of X₁ to X₆ is independently any of oxygen and sulfur, each of Y₁ to Y₆ is independently any of oxygen and sulfur; and each of Q1 and Q2 is independently any of oxygen and sulfur.

The above method is conducive to further improving the stability and capping rate of the mRNA fragments, further reducing the degradation rate of the mRNA fragments in organisms, and further improving translational activity of the mRNA fragments containing the capped polynucleotide when subsequently used for translating proteins.

The inventors found that selection of R₁, R₂ and R₃ may further affect the stability, the capping rate and the translational activity of the mRNA fragments. In some optional embodiments of the present disclosure, in formula I, each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond; each of B₁ to B₄ is independently a nucleobase; m₁ and m₂ are both 1; n₁ is 1, and a sum of n₁, n₂, and n₃ is not greater than 5; each of G1 and G2 is independently selected from hydrogen and methyl; R₁ and R₄ to R₆ are all hydroxyl, R₂ and R₃ are both alkoxy; or R₂ and R₄ to R₆ are all hydroxyl, R₁ and R₃ are both alkoxy; each of X₁ to X₆ is independently any of oxygen and sulfur; each of Y₁ to Y₆ is independently any of oxygen and sulfur; and each of Q1 and Q2 is independently any of oxygen and sulfur.

In the above solution, the selection of R₁, R₂ and R₃ is conducive to further improving the stability and the capping rate of the mRNA fragments, reducing the degradation rate of the mRNA fragments in organisms, and further improving the translational activity of the mRNA fragments containing the capped polynucleotide when subsequently used for translating proteins.

Further, in some optional embodiments of the present disclosure, the capped polynucleotide is a compound of the following general structural formula, or the capped polynucleotide is in a salt form of the compound of the following general structural formula:

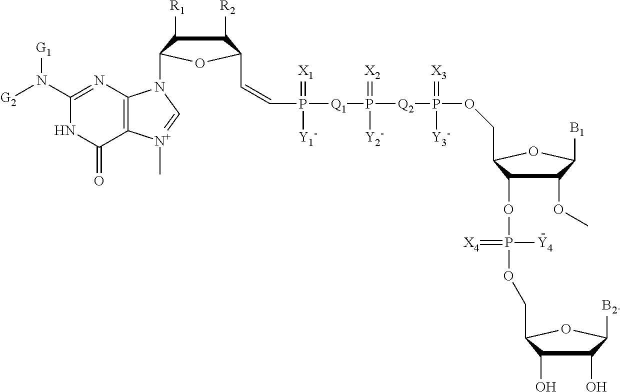

In the above, B₁ and B₂ are nucleobases respectively; each of G1 and G2 is independently selected from hydrogen and methyl; R₁ is hydroxyl, and R₂ is methoxy; or R₂ is hydroxyl, and R₁ is methoxy; each of X₁ to X₄ is independently any of oxygen and sulfur; each of Y₁ to Y₄ is independently any of oxygen and sulfur; and each of Q1 and Q2 is independently any of oxygen and sulfur.

When “R₁ is hydroxyl, R₂ is methoxy; or, R₂ is hydroxyl, and R₁ is methoxy”, it is conducive to further improving the stability and capping rate of the mRNA fragments, further reducing the degradation rate of the mRNA fragments in organisms, and further improving the translational activity of the mRNA fragments containing the capped polynucleotide when subsequently used for translating proteins.

Specifically, in the present embodiment, in the capped polynucleotide of the above general structural formula, G₁ and G₂ are both hydrogen; m₁ and m₂ are both 1; X₁-X₃ and X₅-X₆ are all oxygen, and the whole capped polynucleotide complies with the following general structural formula, or is in a salt form of the compound of the following general structural formula:

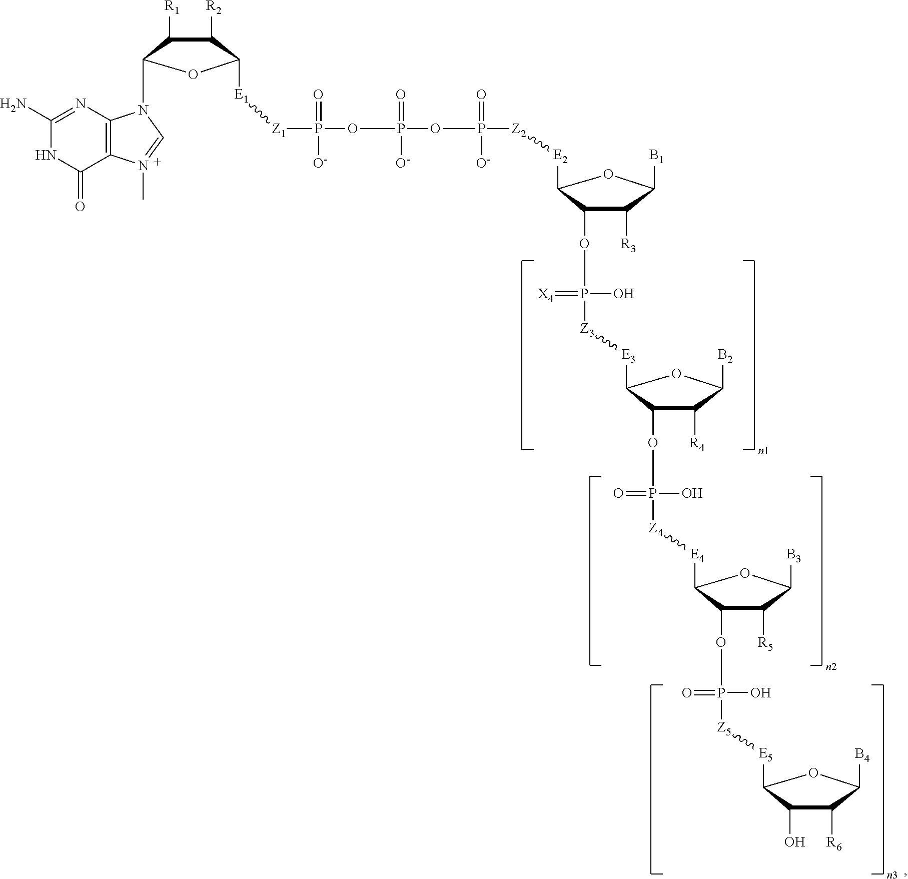

where in this general structural formula, X₄ is oxygen or sulfur; n₁ is 1, and a sum of n₁, n₂, and n₃ is not greater than 5, and each of R₁ to R₆ is independently any of hydrogen, hydroxyl, and methoxy; moreover, each of E₁ to E₅ is carbon, each of Z₁ to Z₅ is independently carbon or oxygen, and at least one pentose and an adjacent phosphate group are linked through carbon-carbon double bonds. The capped polynucleotide complying with this general structural formula can significantly improve the stability of the mRNA fragments, reduce the decomposition rate of the mRNA fragments, and improve the translational activity of the mRNA fragments.

In the above two general structural formulas, due to presence of the carbon-carbon double bonds, a cis configuration (i.e. Z-configuration) and a trans configuration (i.e. E-configuration) necessarily exist, and the inventors find that no matter which carbon-carbon double bond is in the Z-configuration or the E-configuration, the stability and capping rate of the mRNA fragments can be significantly improved, and the degradation rate of the mRNA fragments in organisms can be reduced.

More specifically, in the present embodiment, the capped polynucleotide may be of any of structures in the following table, or in a biologically acceptable salt form of any structure in the following table:

TABLE 1
Specific Structural Formulas of Capped Polynucleotides

| Name | Chemical Structure |
|---|---|
| m⁷G_{2′Ome}VpppA_{2′Ome}pG | 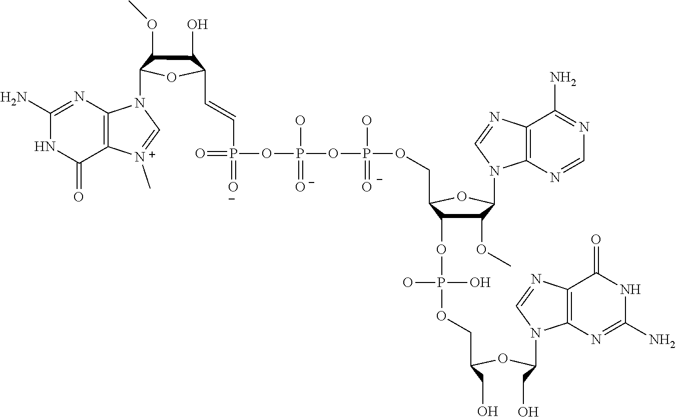 |
| m⁷G_{3′Ome}VpppA_{2′Ome}pG | 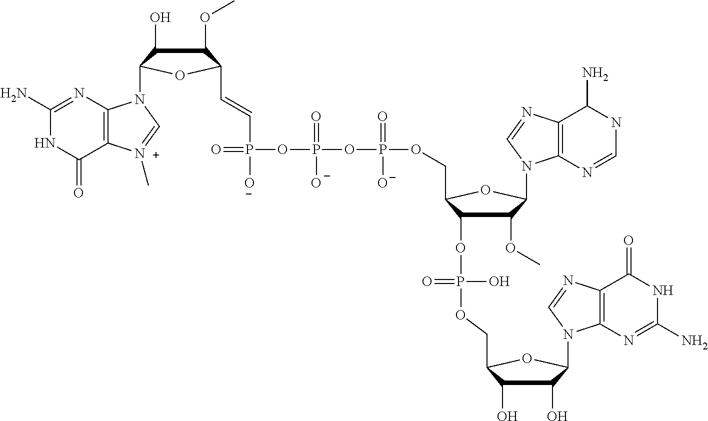 |
| m⁷G_{3′Ome}pppVA_{2′Ome}pG | 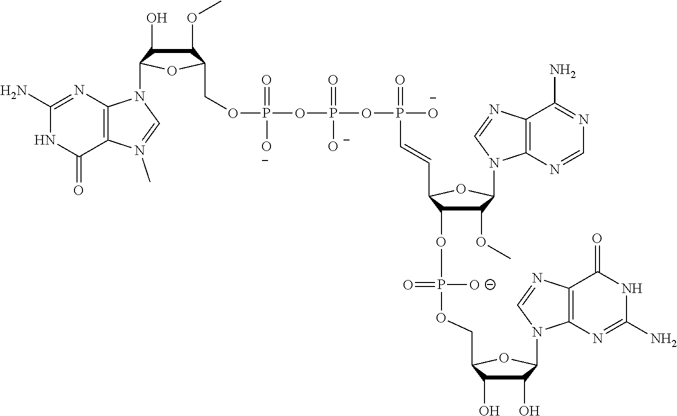 |
| m⁷G_{3′Ome}pppA_{2′Ome}VpG | 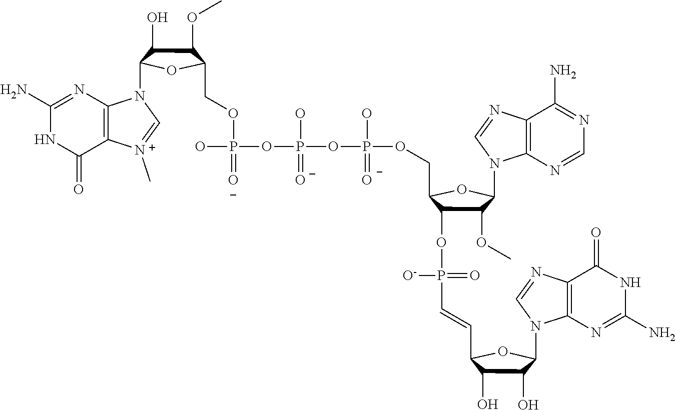 |
| m⁷G_{3′Ome}VpppA_{2′Ome}pC | 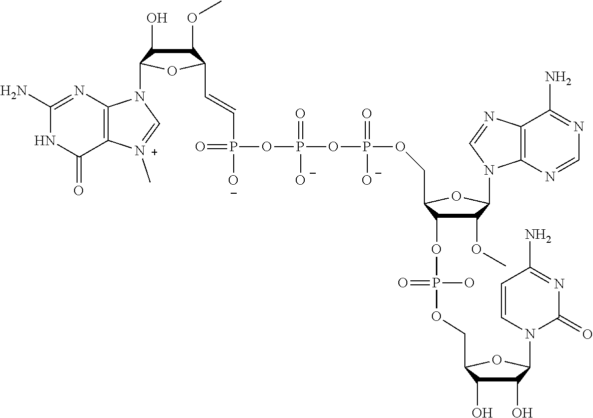 |
| m⁷G_{3′Ome}VpppA_{2′Ome}pU | 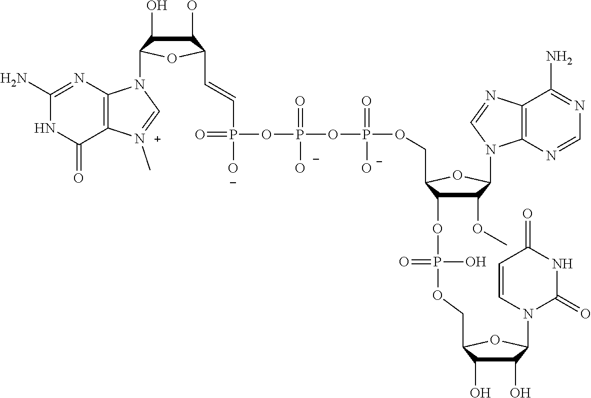 |
| m⁷G_{3′Ome}Vppp(S⁻)A_{2′Ome}pG | 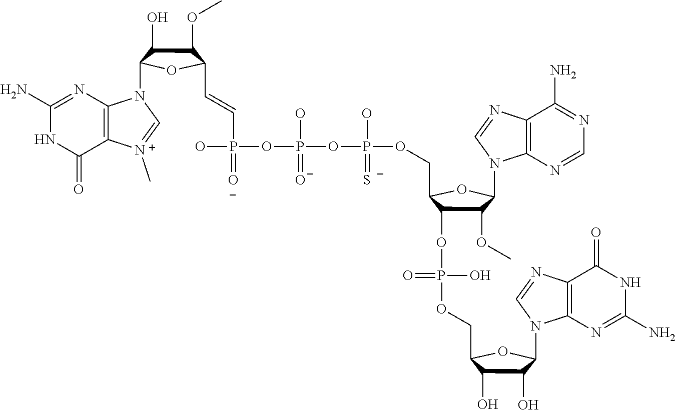 |
| m⁷G_{3′Ome}VpppA_{2′Ome,N}⁶-_{Me}pG | 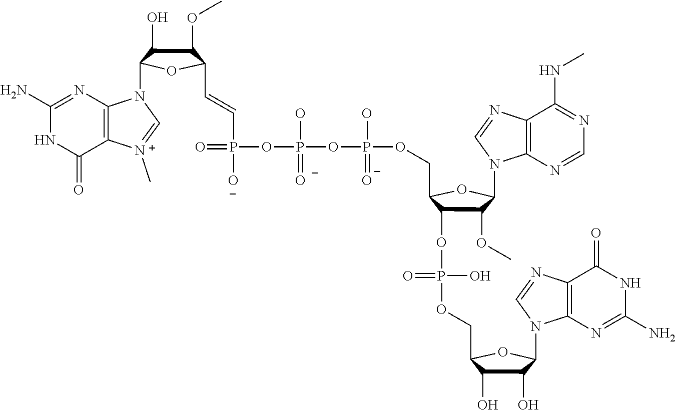 |
| m⁷G_{3′Ome}Vpp(S)pA_{2′Ome,N}⁶-MepG | 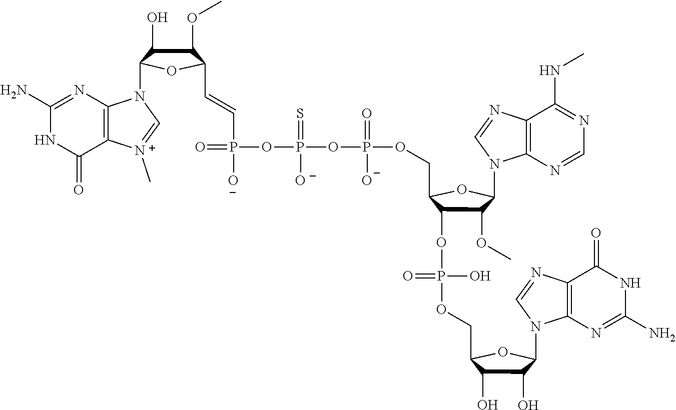 |
| m⁷G_{3′Ome}Vpp(S)pA_{2′Ome}pG | 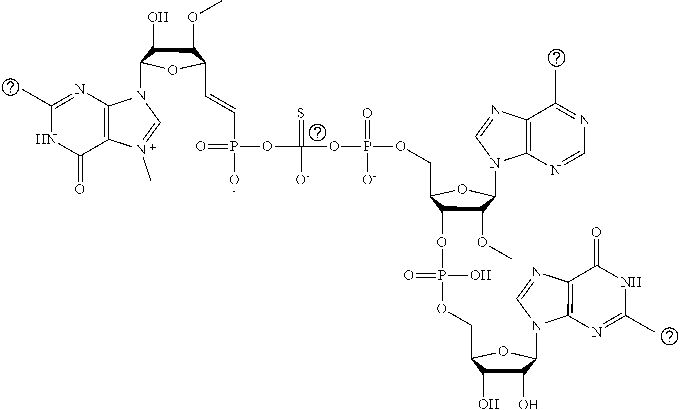 |
| m⁷GVpp(S)pA_{2′Ome}pG | 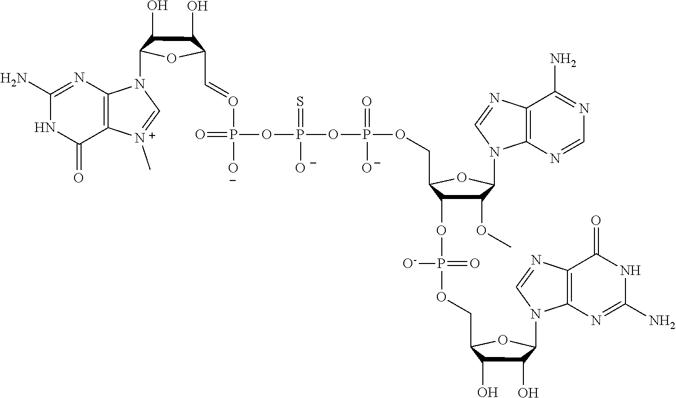 |
| m⁷G_{3′Ome}VppVpA_{2′Ome}pG | 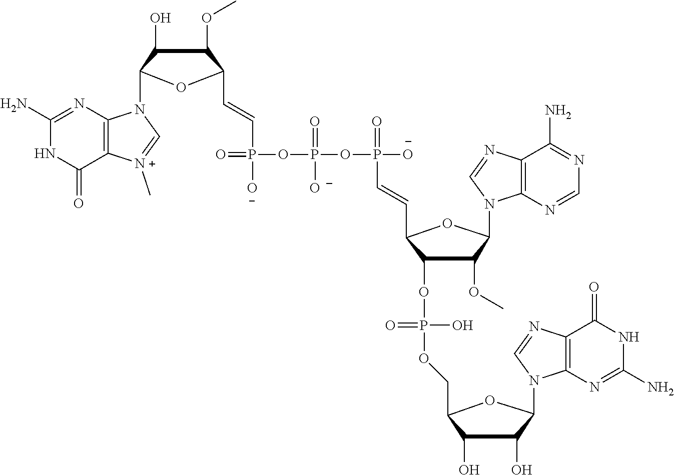 |
| m⁷GVp-S-ppA_{2′Ome}pG | 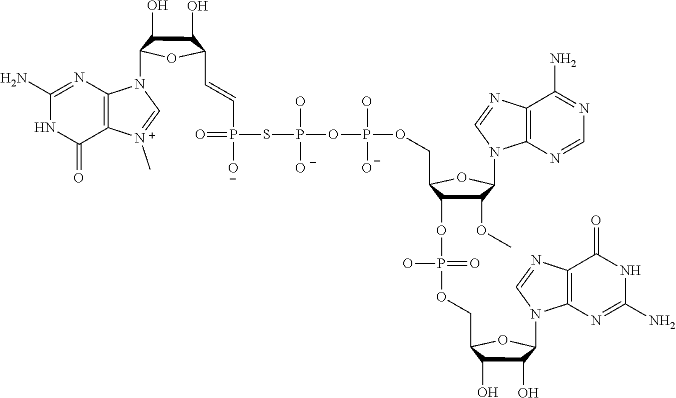 |
| m⁷GVpp-S-pA_{2′Ome}pG | 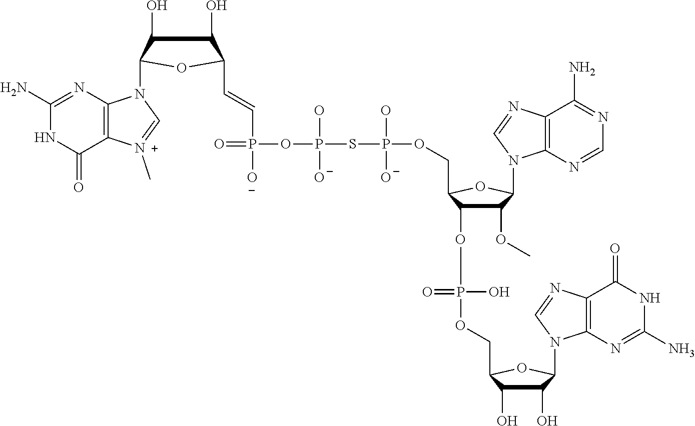 |
| m⁷G_{3′Ome}Vpp(S)pA_{2′Ome}pG | 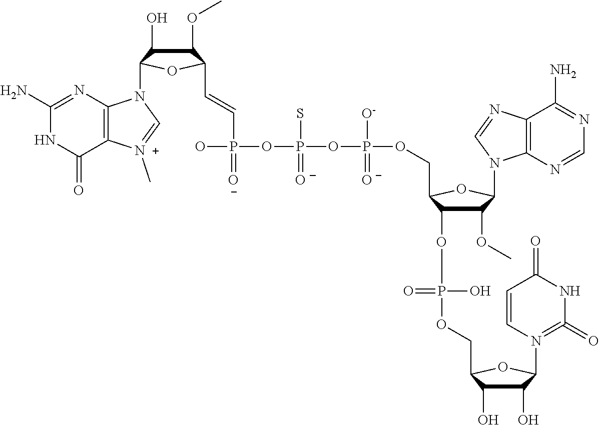 |
| m⁷G_{3′Ome}Vp-S-ppA_{2′Ome}pU | 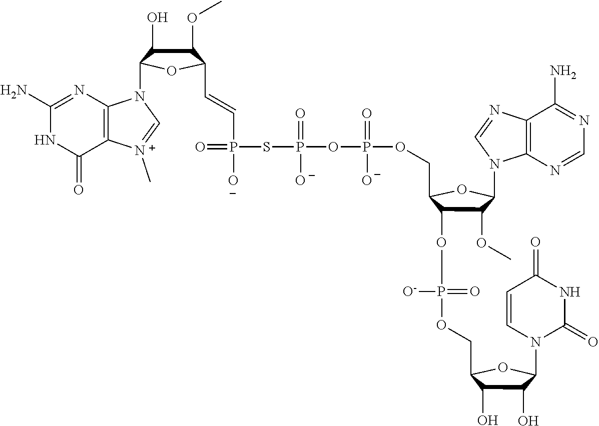 |
| m⁷G_{3′Ome}Vpp-S-pA_{2′Ome}pU | 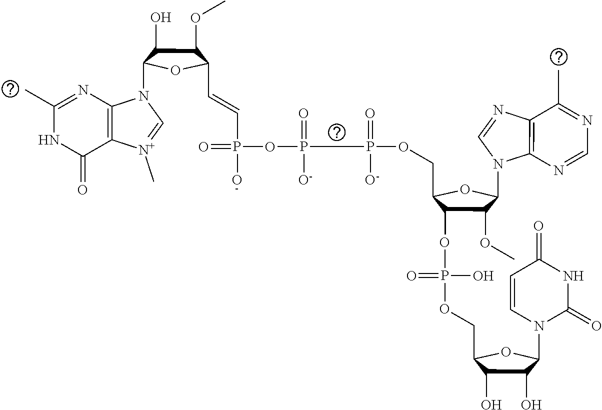 |
| m⁷G_{3′Ome}ppVpA_{2′Ome}pU | 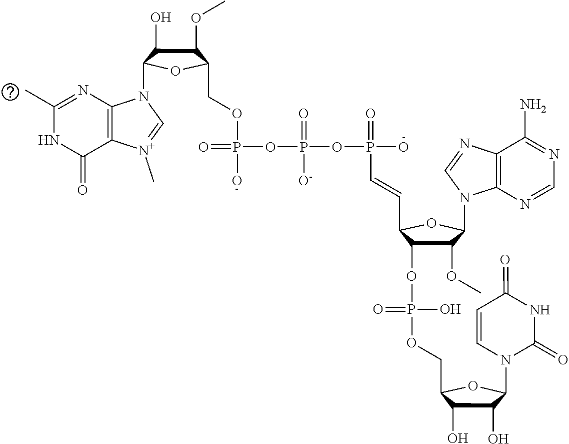 |
| m⁷G_{3′Ome}pppA_{2′Ome}VpU | 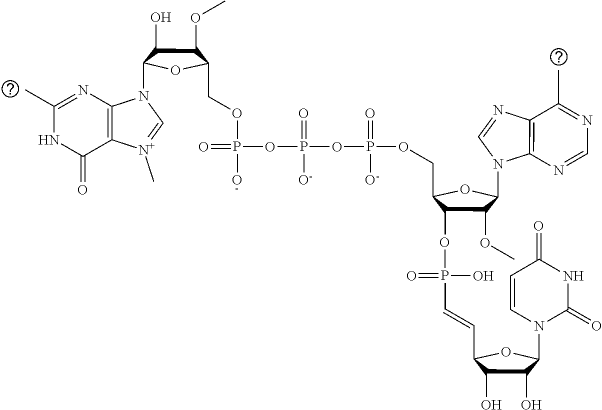 |
| m⁷GVpppA_{2′Ome}VpU | 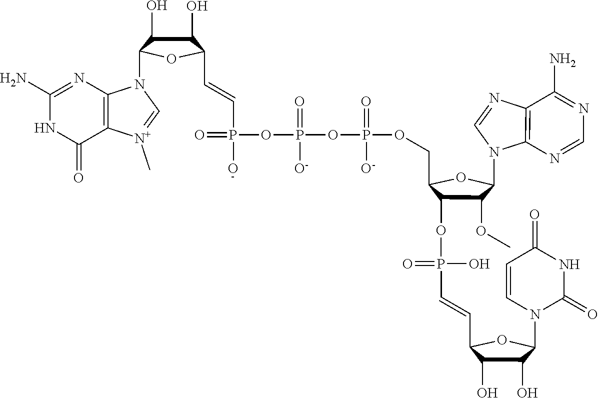 |
| m⁷G_{3′Ome}VpppA_{2′Ome}p(S)U | 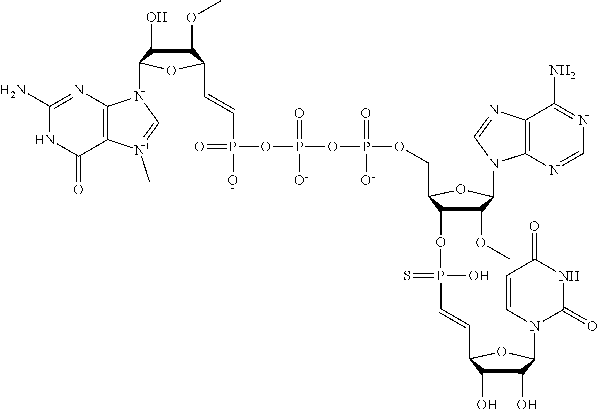 |
| m⁷G_{2′Ome}VpppA_{2′Ome}pGpU | 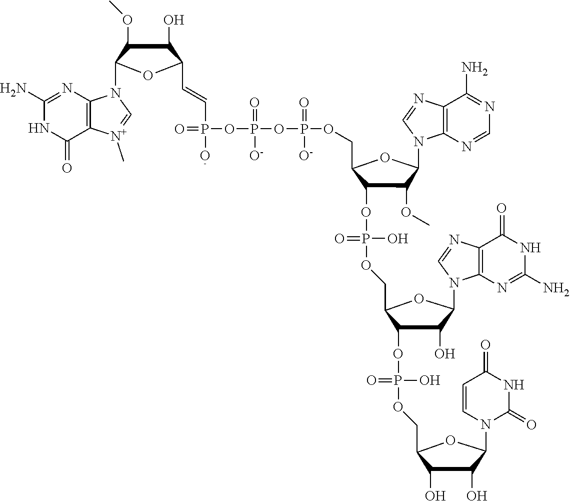 |
| m⁷G_{3′Ome}VpppA_{2′Ome}pGpU | 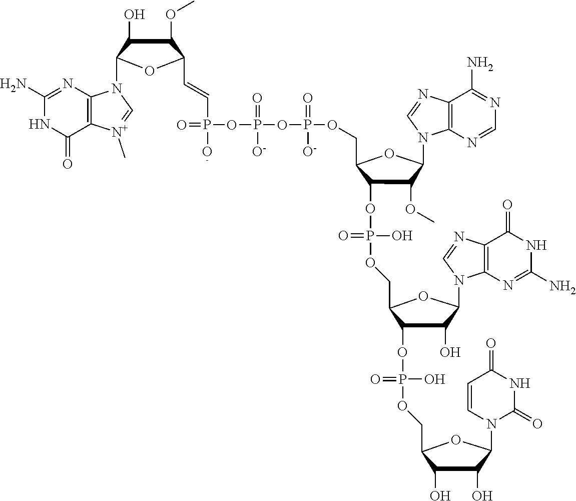 |
| m⁷GVp-S-ppA_{2′Ome}pGpU | 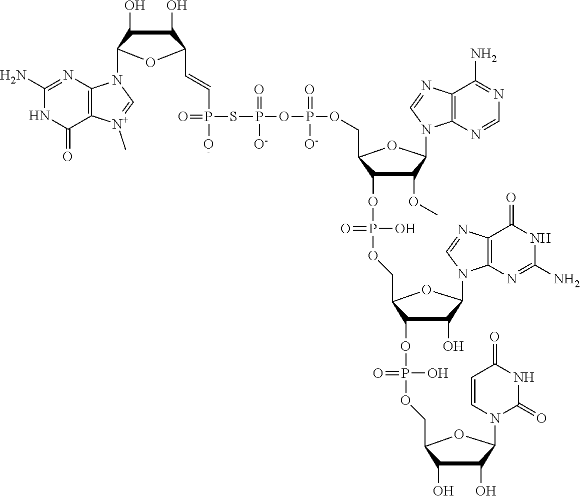 |
| m⁷G_{3′Ome}ppVpA_{2′Ome}pUVpG | 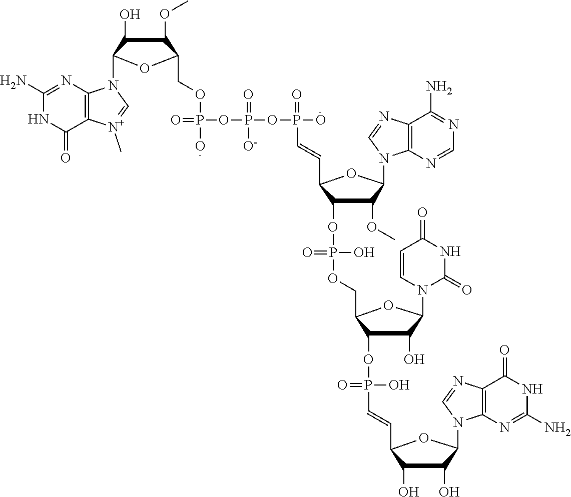 |
| m⁷G_{3′Ome}Vpp-S-pA_{2′Ome}pUpA | 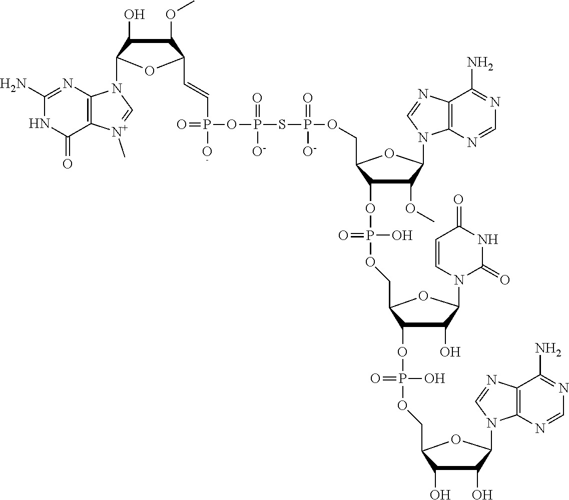 |
| m⁷G_{3′Ome}ppVpA_{2′Ome}pUpA | 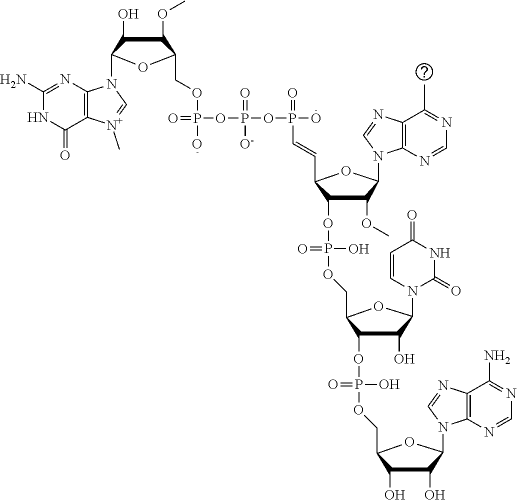 |
| m⁷G_{3′Ome}Vp-S-ppA_{2′Ome}pUpCpA | 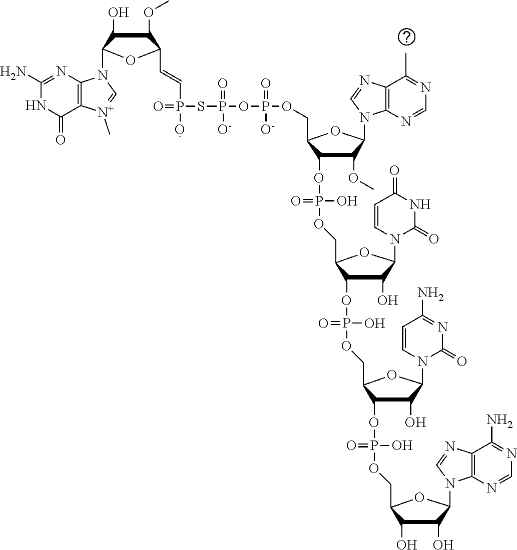 |
| m⁷G_{3′Ome}Vpp-S-pA_{2′Ome}pUpGpA | 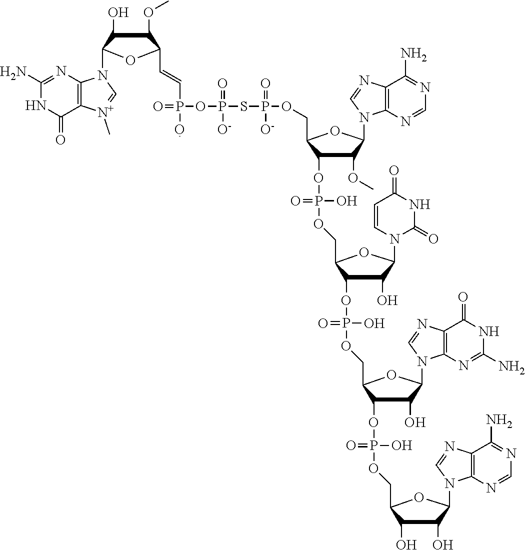 |
| m⁷G_{3′Ome}ppVpA_{2′Ome}pUVpGpA | 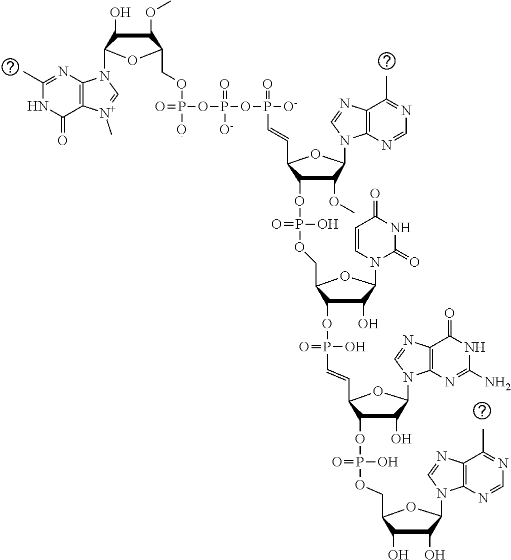 |
| m⁷G_{3′Ome}VpppA_{2′Ome}pGpUVpGpA | 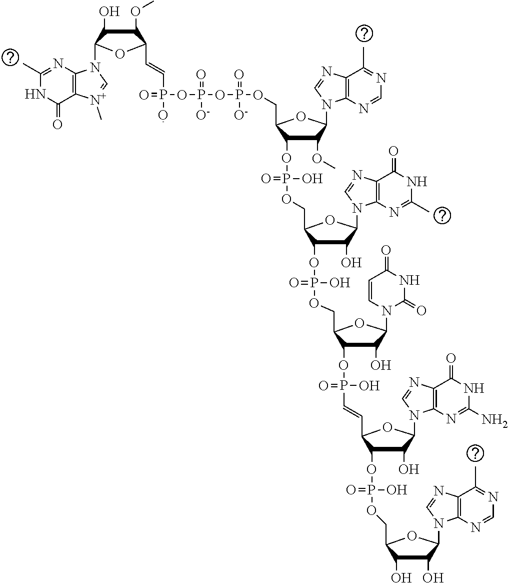 |

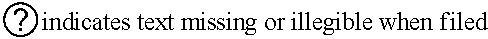

Further, the capped polynucleotide is any of the following compounds, or in a salt form thereof, or a non-salt form thereof:

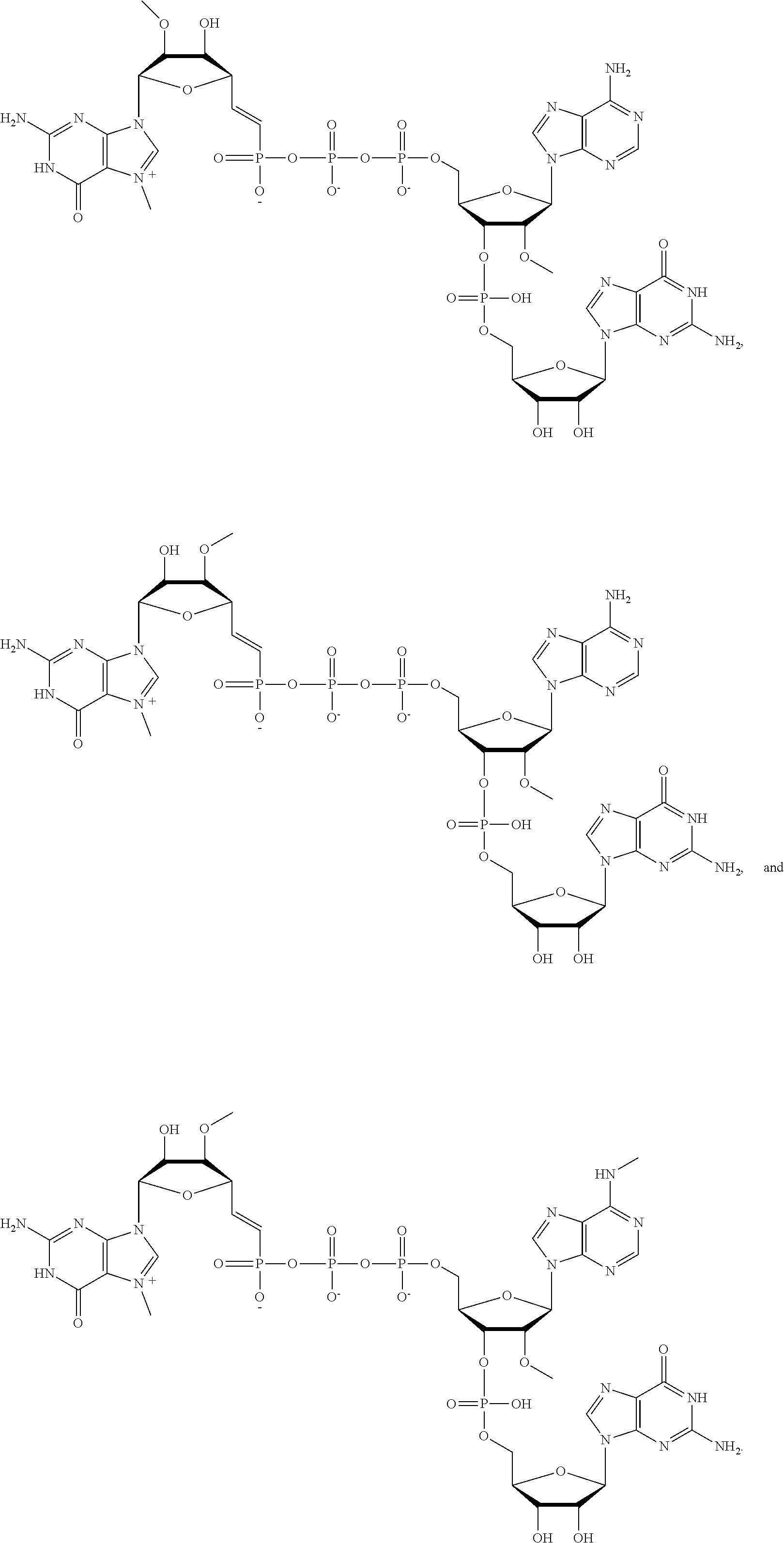

The capped polynucleotide is selected from the compounds of the above structures, which is conducive to further improving the stability and the capping rate of the mRNA fragments, further reducing the degradation rate of the mRNA fragments in organisms, and further improving the translational activity of the mRNA fragments containing the capped polynucleotide when subsequently used for translating proteins.

The above capped polynucleotide is generally synthesized by an oligonucleotide solid phase synthesis process, including following steps:
S100, synthesizing an N7-methylguanosine 5′-Vinyl-5′-diphosphate (m7GDVPIm) intermediate with pentose containing a vinyl phosphonate monomer at a 5′ position;
S20, synthesizing pNpN dinucleotide by a solid-phase synthesizer using raw materials 5′-O-DMT-2′-O-TBDMS phosphoramidite (rA^{Ac} rC^{Ac}, rG^{dmf}, U), 2′-O-MOE-3′-O-phosphoramidite (A_{2′O-TBDMS}^{Ac}, C_{2′O-MOE}^{Ac}, G_{2′O-TBDMS}^{dmf}, U_{2′O-TBDMS}), Bis-cyanoethyl-N,N-diisopropyl CED phosphoramidite, etc.; and
S300, reacting pNpN dinucleotide with phosphor-site imidazolium salt of N7-methylguanosine 5′-Vinyl-5′-diphosphate (m7GVPPIm), to generate the capped polynucleotide.

The applicant will specifically describe in subsequent embodiments a synthesis method by taking m⁷G_{2′Ome}VpppA_{2′Ome} pG (1a for short) and m⁷G_{3′Ome}VpppA_{2′Ome} pG (2a for short) as examples, respectively.

When the above capped polynucleotide is mixed with template DNA, a composition for preparing a capped mRNA can be formed, in which case, the template DNA needs to be designed to contain a transcription site complementary to the capped polynucleotide, so that it can cooperate with the above capped polynucleotide to complete a process of in vitro transcription.

A step of performing transcription to obtain the capped mRNA includes following steps.

In a first step, a template DNA is prepared, so that the DNA template is designed to contain a transcription site complementary to a corresponding capped polynucleotide.

The DNA template in this step is typically formed of a plasmid that becomes linear after a linearization treatment. The plasmid includes a target sequence inserted at a multiple cloning site, and requires a promoter, such as T7, T3 or SP6 promoter, for corresponding polymerase upstream from the target sequence. Moreover, following the promoter, a base complementary to the capped polynucleotide as described above is required as a transcription site.

During the linearization treatment, the DNA can be first linearized through digestion with suitable restriction endonucleases, and then subjected to a purification treatment. Specifically, the restriction endonucleases for the linearization of the plasmid should be able to produce blunt end or 5′-overhang (linearizing the template with enzymes producing 3′-overhang will lead to abnormal transcript), and during the purification treatment, the linearized DNA may be first treated with phenol chloroform, followed by precipitation with ethanol.

In a second step, a composition for preparing the capped mRNA is formulated.

In this step, in addition to the template DNA and the capped polynucleotide, the composition typically further includes mixed enzymes and a reaction buffer, where the reaction buffer includes, but is not limited to, Tris-HCl (pH 7.9), MgCl₂, spermidine, DTT, and Triton X-10.

In a third step, an in vitro transcription reaction is performed, so as to form the capped mRNA.

The capped mRNA prepared includes a main body mRNA fragment and the above capped polynucleotide, 5′ of the main body mRNA fragment being linked to the above capped polynucleotide.

The above capped mRNA can be used for preparing a medicament for treating cancer, and can be used as a vaccine: for example, the capped mRNA can be carried by a vector, where the vector includes any of a lipid nanoparticle, a liposome, a polymer nanoparticle, a solid lipid nanoparticle, and an emulsion; it can also be used for preparing pharmaceutical proteins, for example, the above capped mRNA is used as a template for translation, so as to obtain pharmaceutical proteins.

Characteristics and performances of the present disclosure are further described in detail below in conjunction with examples.

### Example 1

The present example provided a capped polynucleotide, named as 1a. 1a was prepared by reacting intermediate 1a-6 with intermediate 1a-8. During preparation, some intermediates were characterized respectively using a nuclear magnetic resonance spectrometer and a liquid chromatography-mass spectrometer, with specific routes and steps as follows:

(1) Preparation of the intermediate 1a-6, with a route as follows:

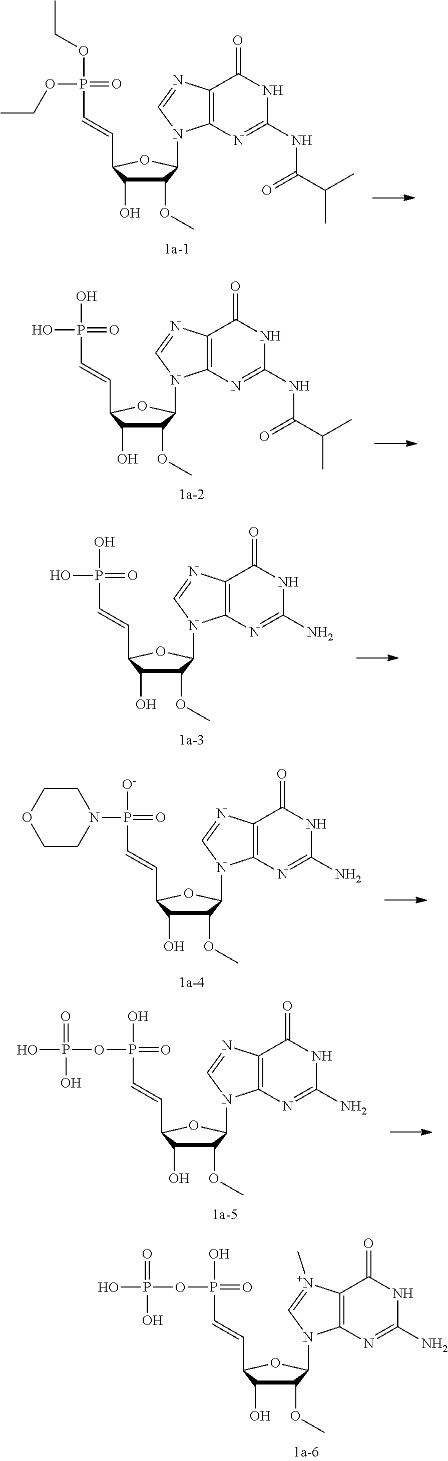

A specific reaction process was as follows:

(1.1) Under nitrogen protection, 7.50 g of compound 1a-1 (1-fold equivalent) was dissolved in 120 ml of DMF (N,N-dimethylformamide). Reaction solution was cooled to 0° C., and 7.2 mL of 2,6-dimethylpyridine and 24.5 mL of TMSBr (bromotrimethylsilane) were added. After the addition, the reaction solution was heated to 50° C., stirred for 12 h, cooled to room temperature, and quenched by adding 120 mL of methanol. The reaction solution was then added with 120 mL of methanol/ammonia water (v:v=1/1), heated to 45° C., stirred for 3 h, cooled to room temperature, and concentrated under reduced pressure to dryness, and a crude product was purified by C18 preparative chromatography, to render compound 1a-3 (5.00 g, 89% yield). Nuclear magnetic resonance data for 1a-1 was: ³¹PNMR (243 MHz, CDCl₃) δ (ppm): 7.98 (s); and LC-MS data were: ESI-MS: m/z 373.1, [M+H]⁺=374.2.

(1.2) Under nitrogen protection, 4.8 g of the compound 1a-3 (1-fold equivalent) was dissolved in 480 mL of tert-butanol/water (v:v=1/1), and added with 0.1 mol/L hydrochloric acid to adjust pH to 2.0. 3.68 g of morpholine (3-fold equivalent) was added, and mixture was stirred at 25° C. for 15 min, heated to 80° C., and refluxed. 8.60 g of DCC (dicyclohexylcarbodiimide, N,N′-dicyclohexylcarbodiimide, 3-fold equivalent) was dissolved in 24 mL of tert-butanol, and resultant was added into the reaction solution in three batches, followed by refluxing at 80° C. for 5 h. The resultant was cooled to room temperature, and filtered, an organic phase was concentrated under reduced pressure to dryness, a crude product was re-dissolved in 100 mL of water, 100 ml of DCM (dichloromethane) was added for extraction and liquid separation, and aqueous phase was concentrated to dryness to render a crude compound 1a-4, which was directly used in the next step without purification.

(1.3) The compound 1a-4 was dissolved in 100 mL of ethanol/acetonitrile (v:v=1/1), resultant was concentrated under reduced pressure to remove water, re-dissolved in 50 mL of DMSO (dimethylsulfoxide), added with 17.6 g of triethylamine phosphate (4-fold equivalent), stirred and reacted at 30° C. for 12 h, cooled to 0° C., and added with 200 mL of water to quench reaction. The reaction solution was purified with a chloride-form resin, to render a compound 1a-5 (2.91 g, 50% yield over two steps). LC-MS data of 1a-5 were: ESI-MS: m/z 453.0, [M+H]⁺=453.1.

(1.4) Under nitrogen protection, 2.91 g of the compound 1a-5 (1-fold equivalent) was dissolved in 30 mL of water, adjusted to pH 4.3 with acetic acid, added with 0.81 g of dimethyl sulfate (6-fold equivalent), and stirred at 25° C. for 20 h. Reaction solution was extracted once by adding 50 mL of DCM, and aqueous phase was purified with the chloride-form resin to render a compound 1a-6 (2.16 g, yield: 72%).

Hydrogen spectrum parameters of 1a-6 were: ¹H NMR (500 MHz, D₂O) δ (ppm): 9.01 (s, 1H), 6.66-6.57 (m, 1H), 6.19 (t, 1H, J=20.0 Hz), 6.07 (d, 1H, J=3.50 Hz), 4.63 (m, 1H), 4.41-4.37 (m, 2H), 4.04 (s, 3H), 3.64 (s, 3H); phosphorus spectrum parameters were: ³¹P NMR (243 MHz, D₂O) δ (ppm): 2.67 (d, 1P, P_{α}), −11.17 (d, 1P, P_{β}); and LC-MS data were: ESI-MS: m/z 467.1, [M+H]⁺=468.3.

(2) Preparation of the intermediate 1a-8, with a route as follows:

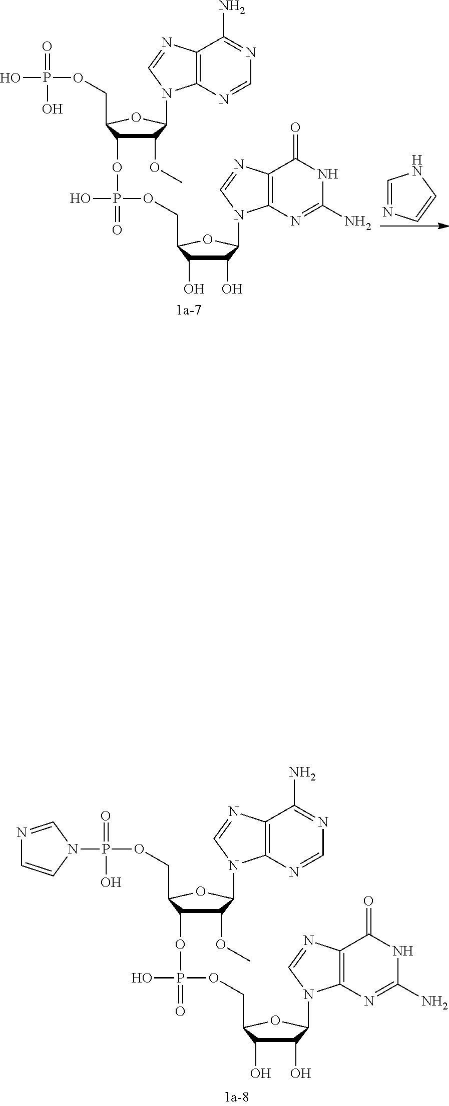

A specific reaction process was as follows:

Under nitrogen protection, 20.0 g of the compound 1a-7 (1-fold equivalent) was dissolved in 300 mL of DMSO. Reaction solution was heated to 38° C., and added with 3.0 g of imidazole (10-fold equivalent), 4.0 g of 2,2-dithiodipyridine (3-fold equivalent), 4.8 g of triphenylphosphine (3-fold equivalent) and 1.9 mL of triethylamine (3-fold equivalent). After the addition, the reaction solution was stirred and reacted at 38° C. for 2 h, and then it is added into an acetone solution (3,000 mL, containing 2% of sodium perchlorate, w/v), stirred for 1 h, and filtered. Solid was concentrated under reduced pressure to render the compound 1a-8 (18 g, yield: 90%). LC-MS data of 1a-8 were: ESI-MS: m/z 756.2, [M+H]⁺=757.3.

(3) Preparation of 1a with 1a-6 and 1a-8, with a reaction route as follows:

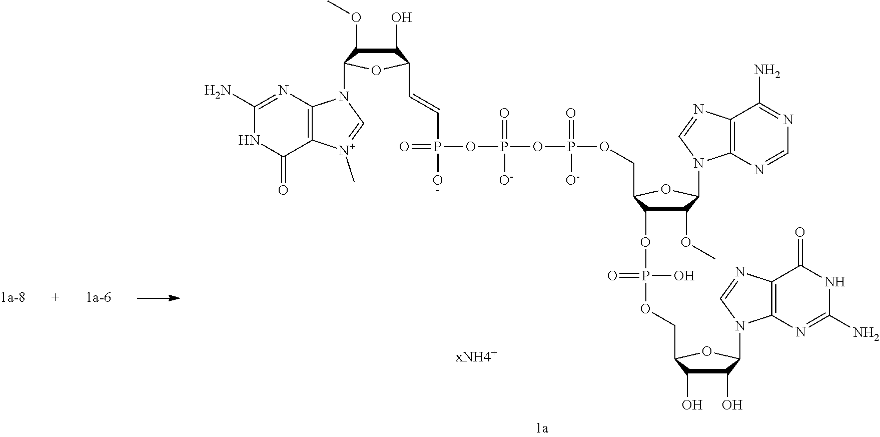

A specific reaction process was as follows: under nitrogen protection, 1 g of the compound 1a-8 (1-fold equivalent) and 2 g of the compound 1a-6 (2.3-fold equivalent) were dissolved in 40 mL of DMSO, added with 1 g of anhydrous magnesium chloride (8-fold equivalent), and stirred and reacted at 25° C. for 2 h, and reaction solution was added into 200 mL of H₂O. The reaction solution was filtered and then purified with a chloride-form resin to render a crude compound 1a, which was purified by the C18 preparative chromatography, to render the compound 1a (0.5 g, yield: 16%). Hydrogen spectrum data of 1a were: ¹H NMR (500 MHz, D₂O) δ (ppm): 8.77 (s, 1H), 8.25 (s, 1H), 7.99 (s, 1H), 7.84 (s, 1H), 6.72-6.68 (m, 1H), 6.64-6.55 (m, 1H), 6.29-6.19 (m, 1H), 5.87 (d, 1H, J=4.0 Hz), 5.73 (t, 2H, J=4.0 Hz), 4.40-4.38 (m, 3H), 4.30-4.28 (m, 3H), 4.12-4.07 (m, 6H), 3.92 (s, 3H), 3.42 (s, 3H), 3.32 (s, 3H); phosphorus spectrum data were: ³¹P NMR (243 MHz, D₂O) δ (ppm): 3.92 (d, 1P, P_{α}), −11.73 (m, 1P, P_{β}), −23.14 (q, 1P, P_{γ}), −0.92 (s, 1P); and LC-MS data were: ESI-MS: m/z 1155.2, [M+H]⁺=1155.4.

### Example 2

The present example provided a capped polynucleotide, named as 2a, 2a was prepared by reacting intermediate 2a-11 with intermediate 2a-13. During preparation, some intermediates were characterized respectively using a nuclear magnetic resonance phosphorus spectrometer and a liquid chromatography-mass spectrometer, with specific routes and steps as follows:

I. Preparation of the intermediate 2a-11, with a synthesis route as follows:

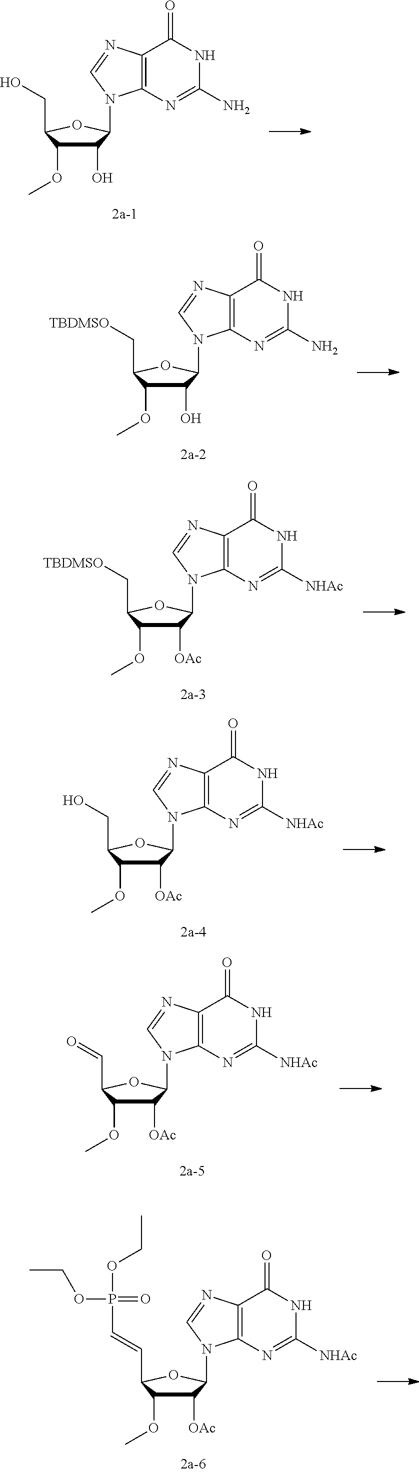

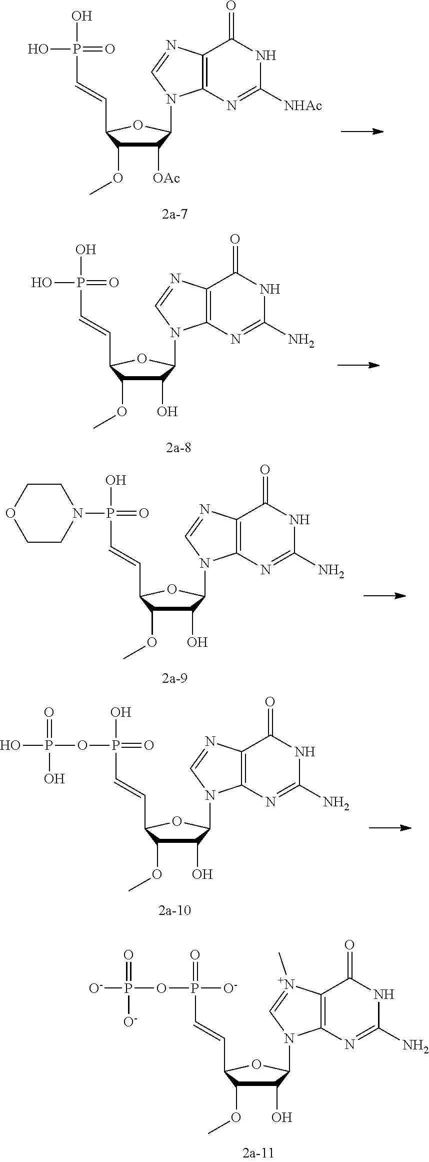

A specific reaction process was as follows.

(1) Under nitrogen protection, 5 g of compound 2a-1 (1.0-fold equivalent) was dissolved in 100 mL of DMF, and then 10 ml of pyridine was added. Reaction solution was cooled to 0° C., then added with 3.8 g of TBDMS-Cl (tert-butyldimethylsilyl chloride), heated to 15° C., and reacted for 20 h. After reaction was ended, the reaction was quenched by adding 2.1 g of sodium bicarbonate and 10 ml of methanol. Then extraction was performed once with 5% sodium bicarbonate aqueous solution and ethyl acetate. Aqueous phase was extracted three times with ethyl acetate. Organic phases were combined, extracted three times with 10% sodium chloride aqueous solution, separated, dried over anhydrous sodium sulfate, and filtered, and filtrate was concentrated to render 7.4 g of crude compound 2a-2.

(2) The above crude compound 2a-2 was dissolved in 74 mL of pyridine, added with 1.1 g of DMAP (4-dimethylaminopyridine, 0.5-fold equivalent) and 7.1 g of acetic anhydride (4-fold equivalent) in sequence, heated to 40° C., and reacted for 10 h. Reaction was stopped. After cooling to room temperature, 5% sodium bicarbonate aqueous solution was added into system until no bubble was generated. Extraction was performed once by adding dichloromethane, an organic phase was separated and extraction was performed once by adding 5% sodium bicarbonate aqueous solution, and an organic phase was separated and then extraction was performed once by adding 10% sodium chloride aqueous solution. The organic phases were separated, dried over anhydrous sodium sulfate, and filtered, and filtrate was concentrated to render 7.1 g of crude compound 2a-3.

(3) The above crude compound 2a-3 was dissolved in 71 ml of pyridine, added with 2.1 g of HF·Py (pyridine hydrofluoride) at 0° C., and heated to 35° C., and reacted for 24 h. Reaction was stopped. Pyridine was removed by high-vacuum concentration. 5 g of crude compound 2a-4 was obtained through the concentration.

(4) Under nitrogen protection, the above crude compound 2a-4 was dissolved in 50 ml of DMSO, added with 8.1 g of DCC (3-fold equivalent), and then added with 1.03 g of pyridine (1-fold equivalent) and 0.75 g of trifluoroacetic acid (0.5-fold equivalent) in sequence to dissolve. Reaction was performed at 25±5° C. for 2.5 h, followed by filtration. Reaction solution was subjected to extraction and liquid separation by adding ethyl acetate and water. Lower aqueous phase was extracted once with ethyl acetate. Organic phases were combined, and washed once with saturated brine. After the liquid separation, upper organic phase was dried over anhydrous sodium sulfate. Then, 5 g of crude compound 2a-5 was obtained through filtration and concentration.

(5) Under nitrogen protection, 5.67 g of tetraethyl methylenediphosphate (1.5-fold equivalent) was dissolved in 50 ml of tetrahydrofuran, cooled to −60±5° C., then added with 5.67 g of sodium tert-pentoxide (1.6-fold equivalent), and stirred at −60±5° C. for 1 h. Then 5 g of the crude compound 2a-5 was dissolved in 50 ml of tetrahydrofuran, and dropwise added into the above reaction system, with a temperature being kept at −60±5° C. during the dropwise addition. After the dropwise addition, resultant was further stirred at −60±5° C. for 1 h, then slowly heated to room temperature, and further stirred for 8 h. Reaction was ended. A saturated NH₄Cl solution was added and mixture was stirred for 10-15 min, and then ethyl acetate was added for extraction and liquid separation. Lower aqueous phase was extracted once again with ethyl acetate. Upper organic phases were combined, and washed once with saturated NH₄Cl and saturated NaCl solution in sequence, to separate the upper organic phases, which were dried over anhydrous sodium sulfate, filtered and then concentrated, to render 4.3 g of crude product. The crude product was purified by the C18 preparative chromatography, to render a compound 2a-6 (2.00 g, overall yield: 23%). ESI-MS: m/z 513.2, [M+H]⁺=514.3.

(6) Under nitrogen protection, 10 g of the compound 2a-6 (1-fold equivalent) was dissolved in 150 mL of DMF. Reaction solution was cooled to 0° C., and added with 9.36 mL of 2,6-dimethylpyridine and 31.85 mL of TMSBr. After the addition, the reaction solution was heated to 50° C., stirred for 12 h, then cooled to room temperature, and quenched by adding 150 mL of methanol. The reaction solution was further added with 150 mL of methanol/ammonia water (v/v=1/1), heated to 45° C., and stirred for 3 h, cooled to room temperature, and concentrated under reduced pressure to dryness. A crude product was purified by the C18 preparative chromatography, to render compound 2a-8 (5.81 g, yield: 80%). Phosphorus spectrum data of 2a-8 were: ³¹P NMR (243 MHz, CDCl3) δ (ppm): 7.91 (s); LC-MS data were: ESI-MS: m/z 373.1. [M+H]⁺=374.2.

(7) Under nitrogen protection, 5 g of the compound 2a-8 (1-fold equivalent) was dissolved in 500 mL of tert-butanol/water (v:v=1/1), and added with 0.1 mol/L hydrochloric acid to adjust pH to 2.0. 3.83 g of morpholine (3-fold equivalent) was added, and mixture was stirred at 25° C. for 15 min, heated to 80° C., and refluxed. 8.96 g of DCC (3-fold equivalent) was dissolved in 25 mL of tert-butanol, and added into the reaction solution in three batches, followed by refluxing at 80° C. for 5 h. The resultant was cooled to room temperature, and filtered, an organic phase was concentrated under reduced pressure to dryness, a crude product was re-dissolved in 100 mL of water, 100 ml of DCM was added for extraction and liquid separation, and aqueous phase was concentrated to dryness to render a crude compound 2a-9, which was directly used in the next step without purification.

(8) The compound 2a-9 was dissolved in 100 mL of ethanol/acetonitrile (v:v=1/1), resultant was concentrated under reduced pressure to remove water, re-dissolved in 50 mL of DMSO, added with 18.3 g of triethylamine phosphate (4-fold equivalent), stirred and reacted at 30° C. for 12 h, and cooled to 0° C., and added with 200 mL of water to quench reaction. The reaction solution was purified with the chloride-form resin, to render a compound 2a-10 (3.0 g, 51% yield over two steps). LC-MS data of 2a-10 were: ESI-MS: m/z 453.0, [M+H]⁺=453.1.

(9) Under nitrogen protection, 3.0 g of the compound 2a-10 (1-fold equivalent) was dissolved in 30 mL of water, adjusted to pH 4-4.5 with acetic acid, added with 0.84 g of dimethyl sulfate (6-fold equivalent), and stirred at 25° C. for 20 h. Reaction solution was extracted once by adding 50 mL of DCM, and aqueous phase was purified with the chloride-form resin to render a compound 2a-11 (2.5 g, yield: 80%). Hydrogen spectrum data of 2a-11 were. ¹H NMR (500 MHz, D₂O) δ (ppm): 9.12 (s, 1H), 6.59-6.42 (m, 1H), 6.05 (t, 1H, J=20.0 Hz), 5.91 (d, 1H, J=3.50 Hz), 4.50 (m, 1H), 4.35-4.28 (m, 2H), 3.95 (s, 3H), 3.59 (s, 3H); phosphorus spectrum data were: ³¹P NMR (243 MHz, D₂O) δ (ppm): 2.61 (d, 1P, P_{α}), −11.10 (d, 1P, P_{β}); and LC-MS data were ESI-MS: m/z 467.1, [M+H]+=468.3.

II. Preparation of the intermediate 2a-13, with a route as follows:

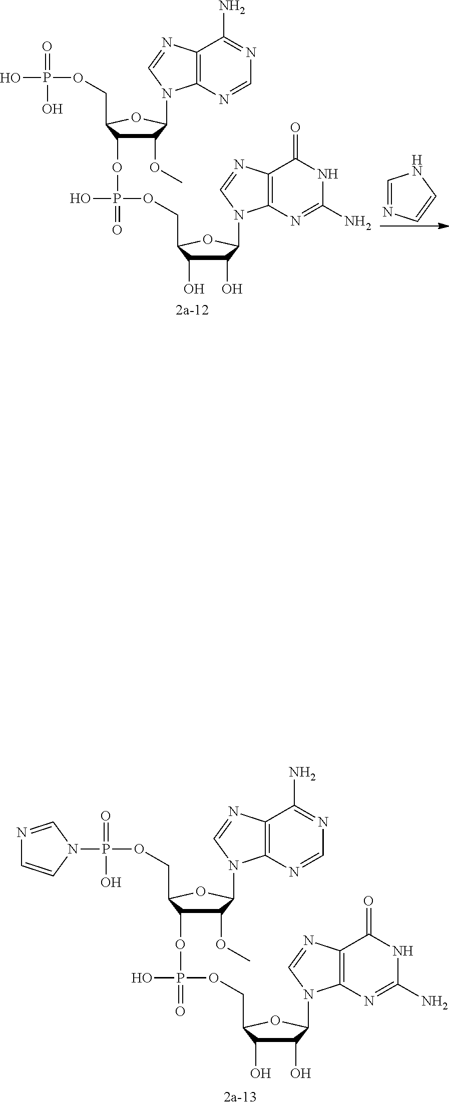

A specific reaction process was as follows: under nitrogen protection, 20.0 g of the compound 2a-12 (1-fold equivalent) was dissolved in 300 mL of DMSO. Reaction solution was heated to 38° C., and added with 3.0 g of imidazole (10-fold equivalent), 4.0 g of 2,2-dithiodipyridine (3-fold equivalent), 4.8 g of triphenylphosphine (3-fold equivalent) and 1.9 mL of triethylamine (3-fold equivalent). After the addition, reaction solution was stirred and reacted at 38° C. for 2 h, and then added into an acetone solution (3,000 mL, containing 2% of sodium perchlorate, w/v), stirred for 1 h and filtered. Solid was concentrated under reduced pressure, to render the compound 2a-13 (18 g, yield: 90%). LC-MS data of 2a-13 were: ESI-MS: m/z 756.2. [M+H]⁺=757.3.

III. Preparation of 2a with 2a-9 and 2a-13, with a reaction route as follows:

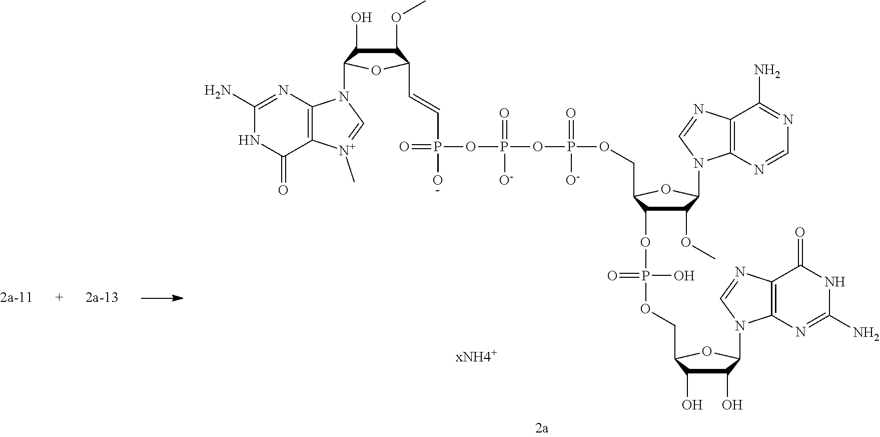

A specific reaction process was as follows: under nitrogen protection, 1 g of compound 2a-13 (I-fold equivalent) and 2 g of the compound 2a-11 (2.3-fold equivalent) were dissolved in 40 mL of DMSO, added with 1 g of anhydrous magnesium chloride (8-fold equivalent), and stirred and reacted at 25° C. for 2 h. Reaction solution was added into 200 mL of H₂O. The reaction solution was filtered and then purified with the chloride-form resin to render a crude compound 2a, which was purified by the C18 preparative chromatography, to render the compound 2a (0.5 g, yield: 16%). Hydrogen spectrum data of 2a were: ¹H NMR (500 MHz, D₂O) δ (ppm): 8.9 (s, 1H), 8.45 (s, 1H), 7.79 (s, 1H), 7.70 (s, 1H), 6.75-6.61 (m, 1H), 6.61-6.50 (m, 1H), 6.19-6.08 (m, 1H), 5.89 (d, 1H, J=4.0 Hz), 5.69 (t, 2H, J=4.0 Hz), 4.43-4.40 (m, 3H), 4.33-4.30 (m, 3H), 4.10-4.08 (m, 6H), 3.95 (s, 3H), 3.38 (s, 3H), 3.30 (s, 3H); phosphorus spectrum data were: ³¹P NMR (243 MHz, D₂O) δ (ppm): 3.90 (d, 1P, P_{α}), −11.73 (m, 1P, P_{β}), −23.14 (q, 1P, P_{γ}), −0.92 (s, 1P); and LC-MS data were: ESI-MS: m/z 1155.2, [M+H]⁺=1155.4.

### Experimental Example 1 Detection of Capping Rate

Composition (i.e. IVT reaction system) for preparing capped Fluc mRNA was formulated firstly: 5 mM NTP (nucleotide triphosphate), 5 mM capped polynucleotides to be tested (capped polynucleotides prepared in Examples 1-2 and capped polynucleotides in comparative examples, respectively), 1× buffer, 1 μg/μL of EGFP (Enhanced Green Fluorescent Protein) linearization template, and 20 KU/mL mixed enzyme system.

In the above, the buffer (10×) was formulated through following steps: mixing Tris-HCl (pH=7.9), MgCl₂, spermidine and DTT, to prepare a buffer (10×) containing magnesium ions; in the buffer, concentration of Tris-HCl was 400 mM, concentration of MgCl₂ was 200 mM, concentration of spermidine was 20 mM, and concentration of DTT was 100 mM.

The mixed enzyme system was formulated through a step as follows: mixing T7 RNA polymerase, inorganic pyrophosphatase and a ribonuclease inhibitor to prepare the mixed enzyme system. In the mixed enzyme system, concentration of T7 RNA polymerase was 400 U/μL, concentration of inorganic pyrophosphatase was 0.1 U/μL, and concentration of nuclease inhibitor was 20 U/μL.

The IVT reaction system was reacted at 37° C. for 3 h; after Dnase I degraded template, Fluc mRNA was purified by LiCl precipitation, to render Fluc mRNA product; where NTPs were ATP, GTP, CTP and uridine (uridine triphosphate, i.e. UTP; or N1-methyl-pseudouridine sodium solution, i.e. N1-Me-pUTP).

Depending on the capped polynucleotide and NTPs, the reaction system was divided into four groups, to determine the capping rate. A detection method was as follows: (1) annealing of probe and mRNA: under a condition of 1×RNaseH Reaction Buffer, adding 500 pmol of probe and 100 pmol of the above Fluc mRNA product, mixing them uniformly and then annealing (maintaining 95° C. for 5 min, and then gradually cooling down to 40° C. at a rate of 2° C./min), so as to render a reaction solution. (2) RNaseH enzyme digestion and elution: 100 μL of magnetic beads (Dynabeads MyOne Streptavidin C1, Invitrogen #65002) were washed twice with Wash buffer (5 mM Tris-HCl pH=7.5, 0.5 mM EDTA, 1 M NaCl), and then the above reaction solution was added, after uniform mixing, 10 μL of RNase H enzyme (M0297L) was added, and reaction was performed at 37° C. for 4 h. After the reaction was ended, washing was performed twice with Wash buffer preparation (5 mM Tris-HCl pH=7.5, 0.5 mM EDTA, 1 M NaCl), 50 μL of enzyme-free water was added, mixture was heated to 80° C., and incubated for 3 min, and then centrifuge tube was placed on magnetic grate to elute supernatant. Eluted system was loaded and analyzed using a liquid chromatography-mass spectrometer, a Waters reverse-phase C18 column (2.1×50 mm), with a mobile phase A: 200 mM hexafluoroisopropanol and 8.15 mM triethylamine, pH 7.9, and a mobile phase B: methanol, was used to obtain an integration result, and two types of sequences were obtained according to different molecular weights. One was sequences with a cap structure at 5′ end, and the other was sequences without a cap structure at 5′ end. Capping rate (%)=(M₁/M₂)×100%, where M₁ was total mass spectrometry response intensity of all sequences with the cap structure at 5′ end, and M₂ was total mass spectrometry response intensity of all sequences with and without the cap structure at 5′ end. Yield and test results of the capping rate of Fluc mRNA are as shown in TABLE 1, where “mM” represents “mmol/L” in the above system.

TABLE 1
Yields and Capping Rates of Different IVT Reaction Systems

| | | | Fluc mRNA | |
|---|---|---|---|---|
| | Capped | Selection of | yield of 1 mL | Capping |
| Group | polynucleotide | uridine | IVT System | rate (%) |
| 1 | Comparative | UTP | 5.1 mg | 98.2 |
| | Example | | | |
| 2 | Comparative | N1-me-pseudo UTP | 4.9 mg | 99.8 |
| | Example | | | |
| 3 | Example 1 | UTP | 5.0 mg | 99.1 |
| 4 | Example 1 | N1-me-pseudo UTP | 4.9 mg | 97.3 |
| 5 | Example 2 | UTP | 5.3 mg | 99.2 |
| 6 | Example 2 | N1-me-pseudo UTP | 5.1 mg | 99.5 |

In the above, the structural formula of the comparative examples was as follows:

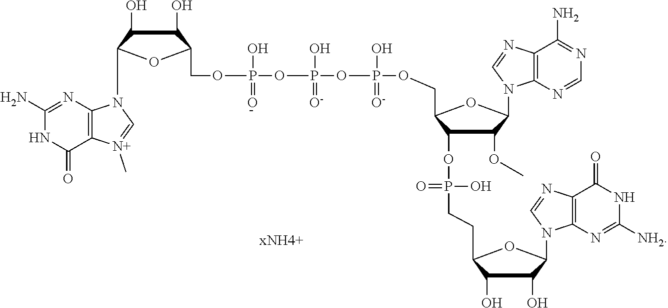

As can be seen from TABLE 1, compared with the capped polynucleotides in the comparative examples, the capped polynucleotides prepared in Examples 1-2 of the present disclosure can improve the capping rate of the mRNA.

### Experimental Example 2 Translation Performance Test

Fluc mRNA products in group 1 to group 6 in Experimental Example 1 were detected for translation performance.

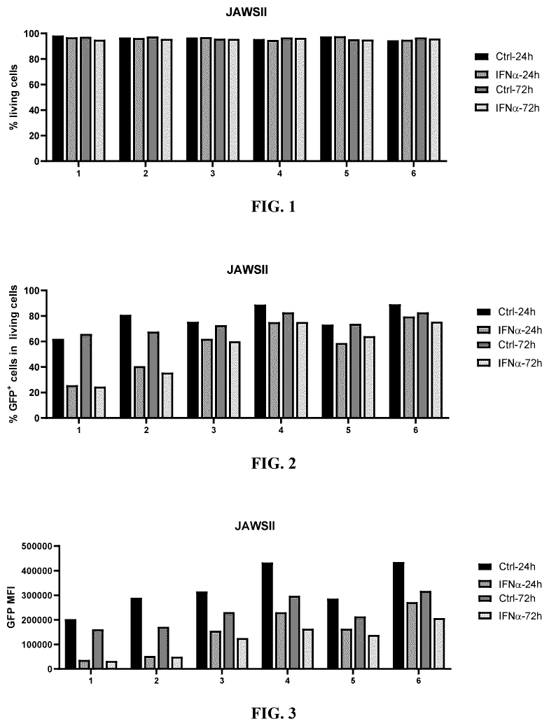

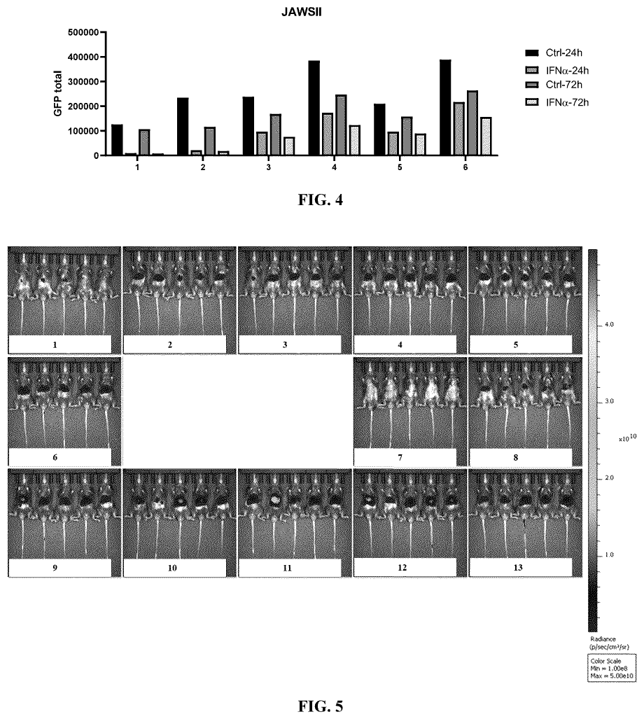

Method for testing the translational performance: Fluc mRNAs synthesized in group 1 to group 6 in Experimental Example 1 were transfected into JAWSII cells of mouse DC cell line. JAWSII cells were plated in a 24-well plate at a density of 2×10⁵/well, and transfection was carried out when the cell density reached approximately 80%. Each well was transfected with 1 μg of Fluc mRNAs, with a transfection reagent Lipofectamine MessengerMAX Transfection Reagent (Invitrogen), following transfection steps according to instructions. In addition, to investigate stability of different cap structures under stress conditions, the present example also treated the cells with 5,000 U/mL IFN α (i.e., interferon α). The cells were pretreated with IFN α for 6 h before the above transfection experiment was carried out. Samples were taken 24 h and 72 h after the transfection, to detect cell viability and GFP expression by flow cytometry, with results as shown in TABLE 2 to TABLE 5 and FIG. 1 to FIG. 4.

TABLE 2
Cell Viability and GFP Expression after 24 h of Transfection of JAWSII without IFN α Treatment

| | | | Cell | GFP positive | | Total |
|---|---|---|---|---|---|---|
| Group | Capped polynucleotide | Selection of uridine | viability (%) | rate (%) | MFI | GFP |
| 1 | Comparative Example | UTP | 98.4 | 62.02 | 202714 | 125723.2 |
| 2 | Comparative Example | N1-me-pseudo UTP | 96.84 | 80.86 | 289614 | 234181.9 |
| 3 | Example 1 | UTP | 96.8 | 75.44 | 315032 | 237660.1 |
| 4 | Example 1 | N1-me-pseudo UTP | 95.61 | 88.85 | 432926 | 384654.8 |
| 5 | Example 2 | UTP | 97.67 | 73.3 | 286035 | 209663.7 |
| 6 | Example 2 | N1-me-pseudo UTP | 94.64 | 89.24 | 434796 | 388012.0 |

TABLE 3
Cell Viability and GFP Expression after 24 h of Transfection of JAWSII with IFN α Treatment

| | | | Cell | GFP positive | | Total |
|---|---|---|---|---|---|---|
| Group | Capped polynucleotide | Selection of uridine | viability (%) | rate (%) | MFI | GFP |
| 1 | Comparative Example | UTP | 96.97 | 25.59 | 36493 | 9338.6 |
| 2 | Comparative Example | N1-me-pseudo UTP | 96.41 | 40.68 | 52746 | 21457.1 |
| 3 | Example 1 | UTP | 97.16 | 62 | 155807 | 96600.3 |
| 4 | Example 1 | N1-me-pseudo UTP | 94.97 | 75.09 | 230502 | 173084.0 |
| 5 | Example 2 | UTP | 97.79 | 58.86 | 163595 | 96292.0 |
| 6 | Example 2 | N1-me-pseudo UTP | 95.01 | 79.64 | 271959 | 216588.1 |

TABLE 4
Cell Viability and GFP Expression after 72 h of Transfection of JAWSII without IFN α Treatment

| | | | Cell | GFP positive | | Total |
|---|---|---|---|---|---|---|
| Group | Capped polynucleotide | Selection of uridine | viability (%) | rate (%) | MFI | GFP |
| 1 | Comparative Example | UTP | 97.41 | 65.96 | 161622 | 106605.9 |
| 2 | Comparative Example | N1-me-pseudo UTP | 97.73 | 67.73 | 171375 | 116072.3 |
| 3 | Example 1 | UTP | 95.88 | 72.9 | 230844 | 168285.3 |
| 4 | Example 1 | N1-me-pseudo UTP | 96.76 | 82.9 | 297893 | 246953.3 |
| 5 | Example 2 | UTP | 95.42 | 73.97 | 213337 | 157805.4 |
| 6 | Example 2 | N1-me-pseudo UTP | 96.86 | 82.89 | 317335 | 263039.0 |

TABLE 5
Cell Viability and GFP Expression after 72 h of Transfection of JAWSII with IFN α Treatment

| | | | Cell | GFP positive | | Total |
|---|---|---|---|---|---|---|
| Group | Capped polynucleotide | Selection of uridine | viability (%) | rate (%) | MFI | GFP |
| 1 | Comparative Example | UTP | 95.03 | 24.55 | 32631 | 8010.9 |
| 2 | Comparative Example | N1-me-pseudo UTP | 95.83 | 35.53 | 49785 | 17688.6 |
| 3 | Example 1 | UTP | 95.75 | 60.11 | 125608 | 75503.0 |
| 4 | Example 1 | N1-me-pseudo UTP | 96.53 | 75.29 | 163641 | 123205.3 |
| 5 | Example 2 | UTP | 95.21 | 64.1 | 138498 | 88777.2 |
| 6 | Example 2 | N1-me-pseudo UTP | 96.06 | 75.53 | 206843 | 156228.5 |

In TABLE 2 to TABLE 5, “MFI” represents mean fluorescence intensity, and “Total GFP” represents total fluorescence signal.

Horizontal coordinates 1 to 6 in FIG. 1 to FIG. 4 respectively represent group 1 to group 6; in FIG. 1 to FIG. 4, “Ctrl-24 h” represents an experimental group without IFN α treatment and with a transfection duration of 24 h; “Ctrl-72 h” represents an experimental group without IFN α treatment and with a transfection duration of 72 h; “IFN α-24 h” represents an experimental group with 6 h of IFN α treatment and then a transfection duration of 24 h; and “IFN α-72 h” represents an experimental group with 6 h of IFN α treatment and then a transfection duration of 72 h.

It can be seen from TABLE 2 to TABLE 5 and FIG. 1 to FIG. 4 that none of the mRNAs prepared with the capped polynucleotides in comparative examples and Examples 1-2 affected the cell viability. Compared with the mRNAs prepared with the capped polynucleotides in the comparative examples, the mRNAs prepared with the capped polynucleotides in Examples 1-2 can enhance expression of GFP. In addition, after the treatment with IFN α, the expression of GFP in the mRNAs prepared with the capped polynucleotide in the comparative examples was remarkably affected, but the expression of GEP in the mRNAs prepared with the capped polynucleotides in Examples 1-2 was less variable. Therefore, compared with the mRNAs prepared with the capped polynucleotides in the comparative examples, the mRNAs prepared with the capped polynucleotides in Examples 1-2 had higher stability and better translation effect.

### Experimental Example 3 Translation Performance Test

Using a PNI ignite device, the Fluc mRNAs prepared in group 1 to group 6 in Experimental Example 1 synthesized in the above were respectively encapsulated at a flow rate of aqueous phase:ethanol phase=3:1, a molar ratio of various lipid components in the ethanol phase being: Dlin-MC3:PEG2000-DMG:DSPC:Cholesterol=50:1.5:38.5:10. After encapsulation, 100 K molecular weight ultrafiltration centrifuge tube was used for exchange of corresponding PBS solution and concentration, to render 0.2 mg/mL Fluc LNP.

Using the PNI ignite device, encapsulation was performed at a flow rate of aqueous phase: ethanol phase=3:1, a molar ratio of various lipid components in the ethanol phase being: Dlin-MC3:PEG2000-DMG:DSPC:Cholesterol=50:1.5:38.5:10. After encapsulation, 100 K molecular weight ultrafiltration centrifuge tube was used for exchange of corresponding PBS solution and concentration, to render 0.2 mg/mL blank LNP.

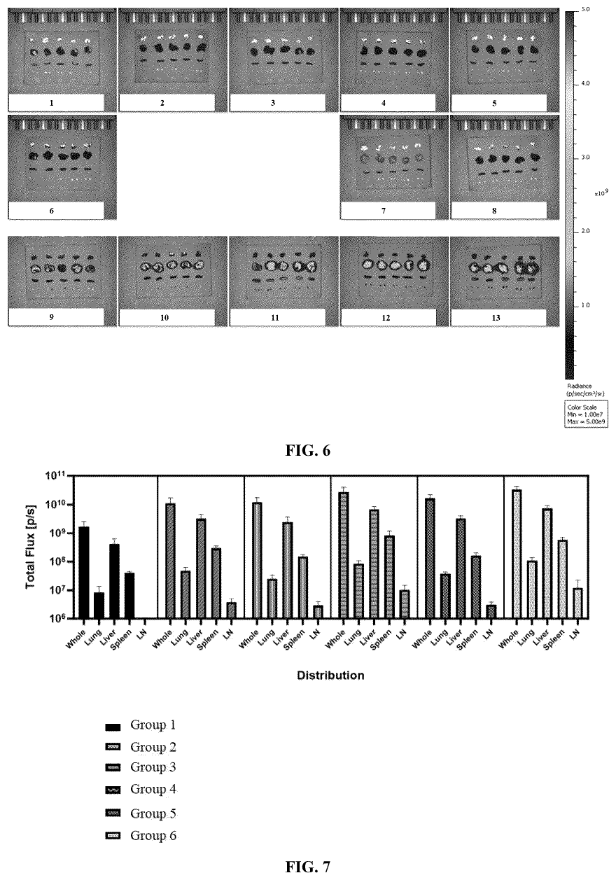

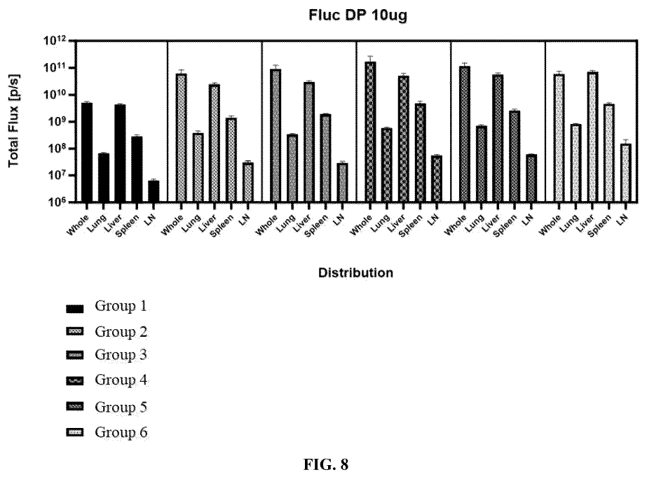

The Fluc LNP (at a dose of 1 μg and 10 μg) and blank LNP (at a dose of 50 μL) prepared in the above were intravenously injected into mice C57. 6 h after injection, the mice were imaged in vivo, and then sacrificed. Organs (lung, liver, spleen, and lymph) of the mice were taken and respectively imaged with an imager, to collect Flux values. In the above, in vivo imaging results of the mice were as shown in FIG. 5, images of organs of the sacrificed mice were as shown in FIG. 6, Flux values of the organs of the mice sacrificed after injection of Fluc NP (at a dose of 1 μg) were as shown in FIG. 7, and Flux values of the organs of the mice sacrificed with injection of Fluc NP (at a dose of 10 μg) were as shown in FIG. 8.

In FIG. 5 and FIG. 6, numbers 1-6 respectively represent Fluc NPs (injection dose being 1 μg) corresponding to group 1 to group 6 in Experimental Example 1, number 7 represents the blank LNP (injection dose being 50 μL), and numbers 8-13 respectively represent Fluc NPs (injection dose being 10 μg) corresponding to group 1 to group 6 in Experimental Example 1. In FIG. 6, the organs in each drawing from top to bottom are: lung, liver, spleen and lymph node, respectively. In FIG. 7 and FIG. 8, horizontal coordinate “Total Flux” represents fluorescence value, Whole represents whole organs, Lung represents lung, Liver represents liver, Spleen represents spleen, and LN represents lymph node.

It can be seen from FIG. 5 to FIG. 8 that the mRNAs prepared with the capped polynucleotides in Examples 1-2, compared with the mRNAs prepared with the capped polynucleotides in comparative examples, had better translation effect.

### INDUSTRIAL APPLICABILITY

The capped polynucleotide, the capped mRNA and composition, the pharmaceutical protein and the preparation method therefor and use thereof, and the pharmaceutical preparation provided in the present disclosure can enhance the stability and capping rate of the capped mRNA, reduce the degradation rate of the capped mRNA in organisms, and improve the translational activity of the capped mRNA.

1. A capped polynucleotide, wherein the capped polynucleotide is a compound of the following general structural formula, or the capped polynucleotide is in a salt form of the compound of the following general structural formula: 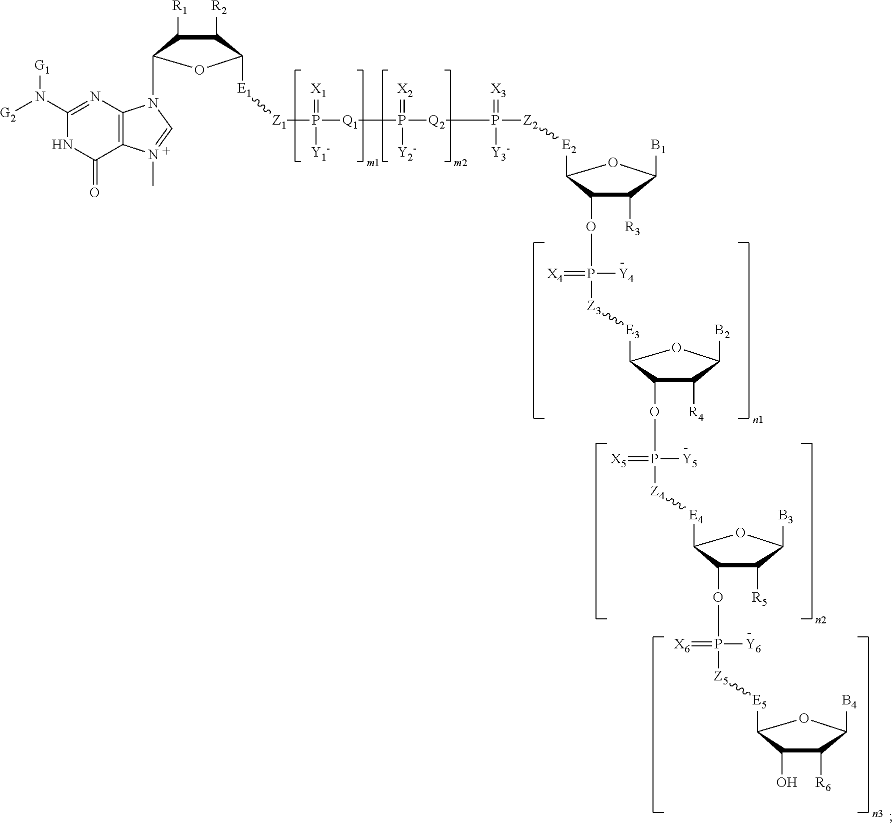
each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond;
each of B₁ to B₄ is independently a nucleobase;
each of m₁ and m₂ is independently 0 or 1;
n₁ is 1, and each of n₂ and n₃ is independently any integer from 0 to 20;
each of G₁ and G₂ is independently selected from hydrogen and methyl;
each of R₁ to R₆ is independently any of hydrogen, hydroxyl, alkyl, alkoxy, and halo;
each of X₁ to X₆ is independently any of oxygen, sulfur, ═NH, and ═NCH₃;
each of Y₁ to Y₆ is independently any of oxygen, sulfur, selenium, aryl, alkyl, alkoxy, aryloxy, —BH₂, —NH, —NCH₃, and —N(C(═O)CH₃); and
each of Q₁ and Q₂ is independently selected from the group consisting of oxygen, sulfur, and methylene.

2. The capped polynucleotide according to claim 1, wherein
each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond;
each of B₁ to B₄ is independently a nucleobase;
each of m₁ and m₂ is independently 0 or 1;
n₁ is 1, and a sum of n₁, n₂, and n₃ is not greater than 5;
each of G₁ and G₂ is independently selected from hydrogen and methyl;
each of R₁ to R₆ is independently any of hydrogen, hydroxyl, and alkoxy;
each of X₁ to X₆ is independently any of oxygen and sulfur;
each of Y₁ to Y₆ is independently any of oxygen and sulfur; and
each of Q₁ and Q₂ is independently any of oxygen and sulfur.

3. The capped polynucleotide according to claim 1, wherein
each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond;
each of B₁ to B₄ is independently a nucleobase;
n₁ is 1, and a sum of n₁, n₂, and n₃ is not greater than 5;
each of m₁ and m₂ is independently 0 or 1;
each of G₁ and G₂ is independently selected from hydrogen and methyl;
each of R₁ to R₆ is independently any of hydroxyl and alkoxy;
each of X₁ to X₆ is independently any of oxygen and sulfur;
each of Y₁ to Y₆ is independently any of oxygen and sulfur; and
each of Q₁ and Q₂ is independently any of oxygen and sulfur.

4. The capped polynucleotide according to claim 1, wherein
each of E₁ to E₅ is carbon, each of Z₁ to Z₅ is independently carbon or oxygen, and at least one position between E₁ and Z₁, between E₂ and Z₂, between E₃ and Z₃, between E₄ and Z₄, and between E₅ and Z₅ is a carbon-carbon double bond;
each of B₁ to B₄ is independently a nucleobase;
m₁ and m₂ are both 1;
n₁ is 1, and a sum of n₁, n₂, and n₃ is not greater than 5;
each of G₁ and G₂ is independently selected from hydrogen and methyl;
R₁ and R₄ to R₆ are all hydroxyl, R₂ and R₃ are both alkoxy; or R₂ and R₄ to R₆ are all hydroxyl, R₁ and R₃ are both alkoxy;
each of X₁ to X₆ is independently any of oxygen and sulfur;
each of Y₁ to Y₆ is independently any of oxygen and sulfur; and
each of Q₁ and Q₂ is independently any of oxygen and sulfur.

5. The capped polynucleotide according to claim 1, wherein the capped polynucleotide is a compound of a following general structural formula, or the capped polynucleotide is in a salt form of the compound of the following general structural formula: 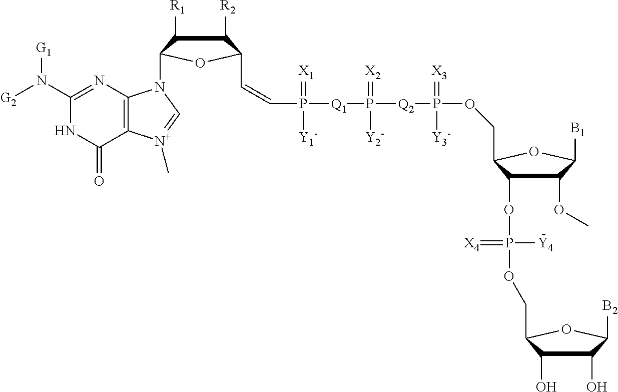
B₁ and B₂ are nucleobases respectively;
each of G₁ and G₂ is independently selected from hydrogen and methyl;
R₁ is hydroxyl, and R₂ is methoxy; or R₂ is hydroxyl, and R₁ is methoxy;
each of X₁ to X₄ is independently any of oxygen and sulfur;
each of Y₁ to Y₄ is independently any of oxygen and sulfur; and
each of Q₁ and Q₂ is independently any of oxygen and sulfur.

6. The capped polynucleotide according to claim 1, wherein the capped polynucleotide is a compound of a following general structural formula, or the capped polynucleotide is in a salt form of the compound of the following general structural formula: 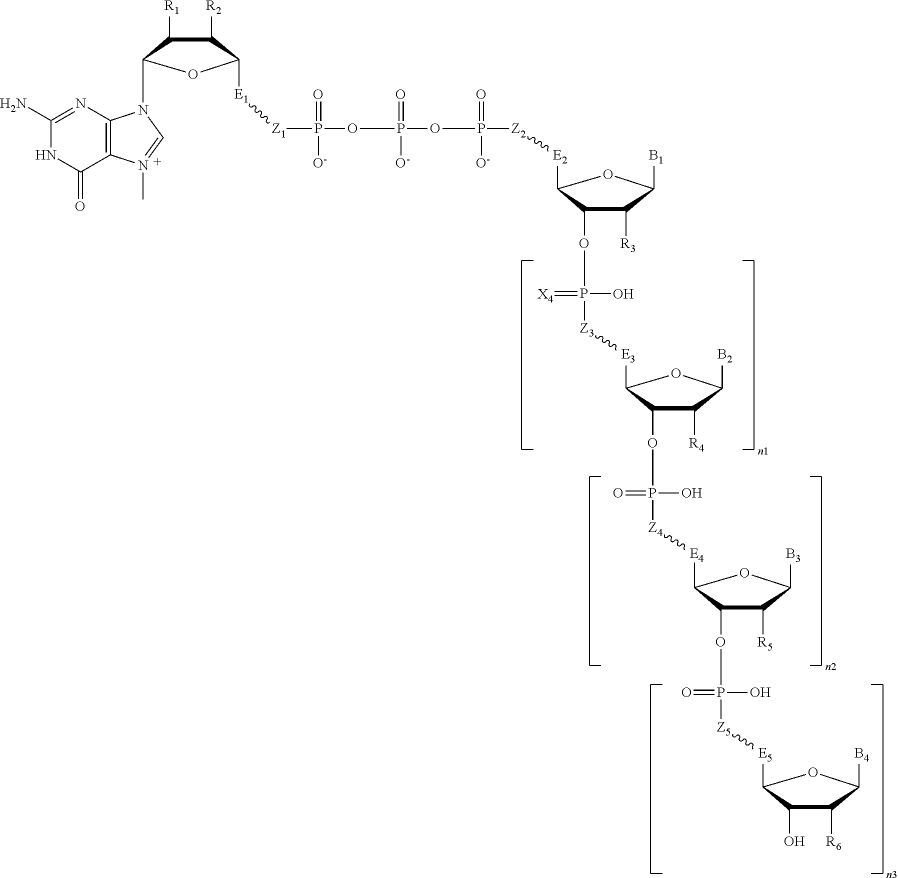
in the above general structural formula, each of E₁ to E₅ is carbon, each of Z₁ to Z₅ is independently carbon or oxygen, and at least one pentose and an adjacent phosphate group are linked through carbon-carbon double bonds;
each of B₁ to B₄ is independently a nucleobase;
n₁ is 1, and a sum of n₁, n₂, and n₃ is not greater than 5;
each of R₁ to R₆ is independently any of hydrogen, hydroxyl, and methoxy; and
X₄ is oxygen or sulfur.

7. The capped polynucleotide according to any one of claims 1-6, wherein the carbon-carbon double bond in the general structural formula are in at least one of Z-configuration and E-configuration.

8. The capped polynucleotide according to any one of claims 1-6, wherein the capped polynucleotide is any of following compounds or in a salt form thereof:

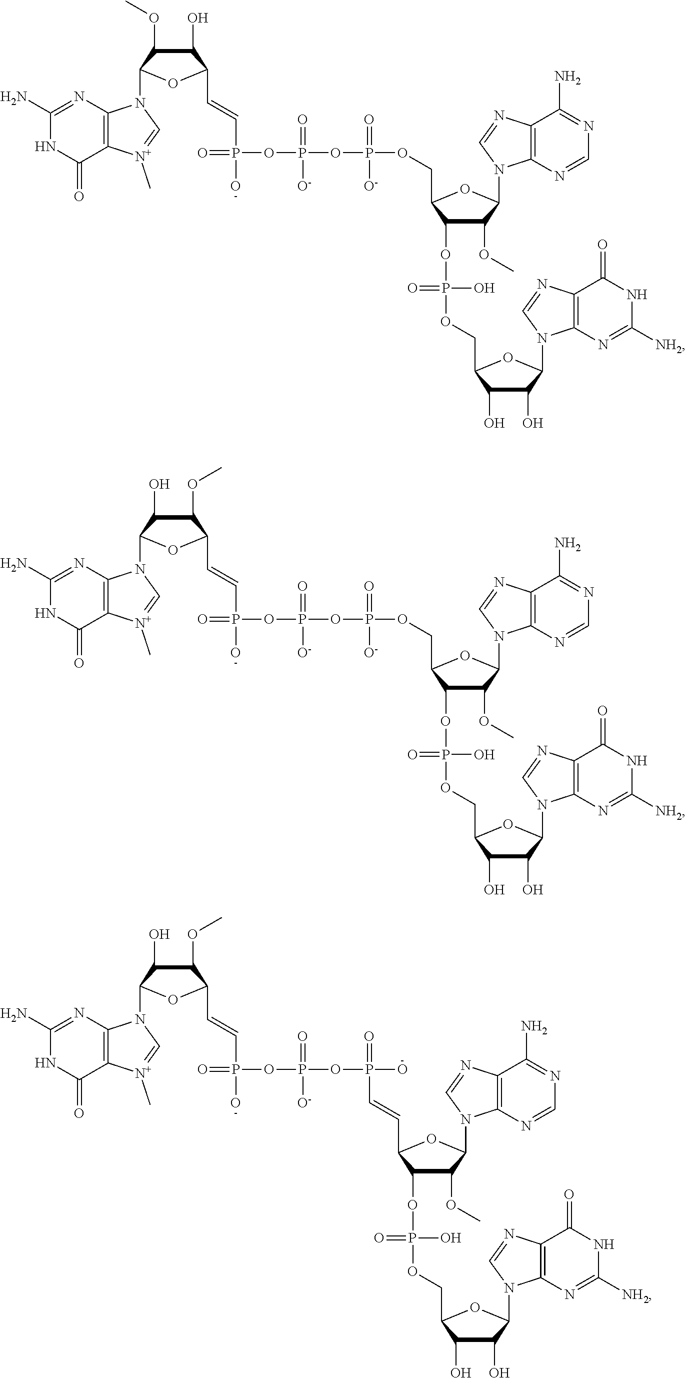

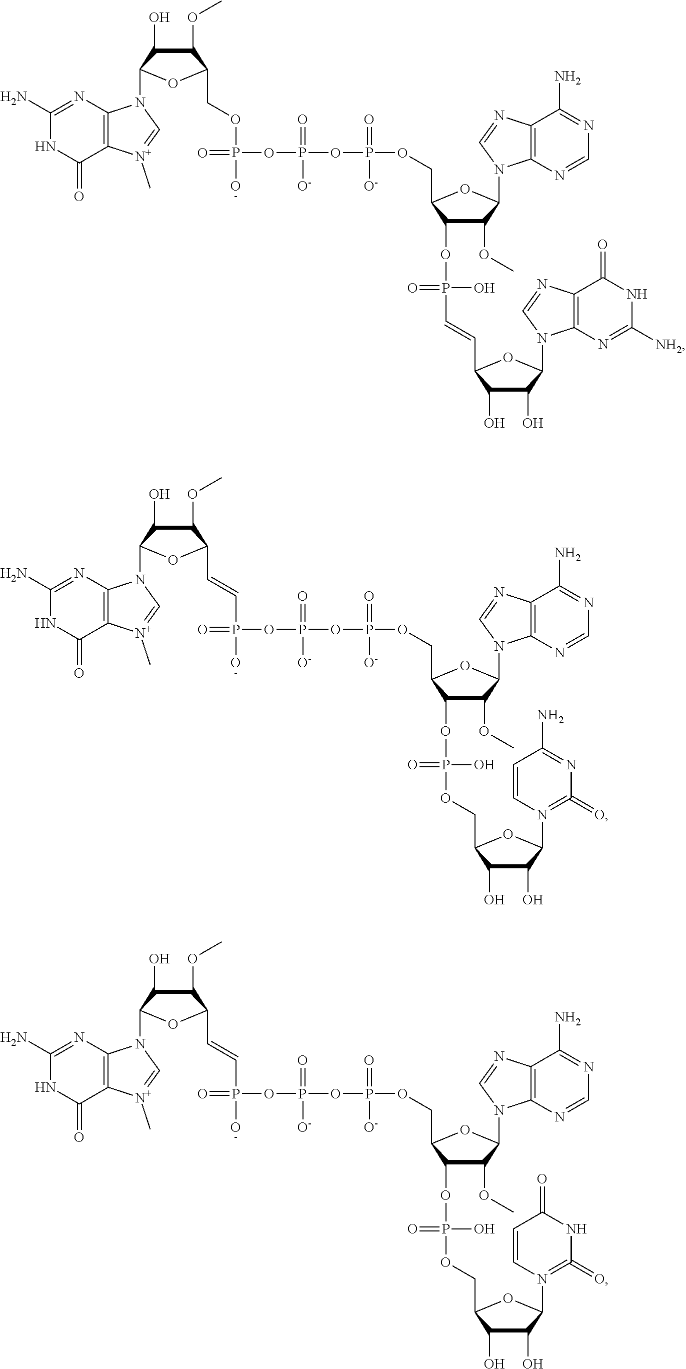

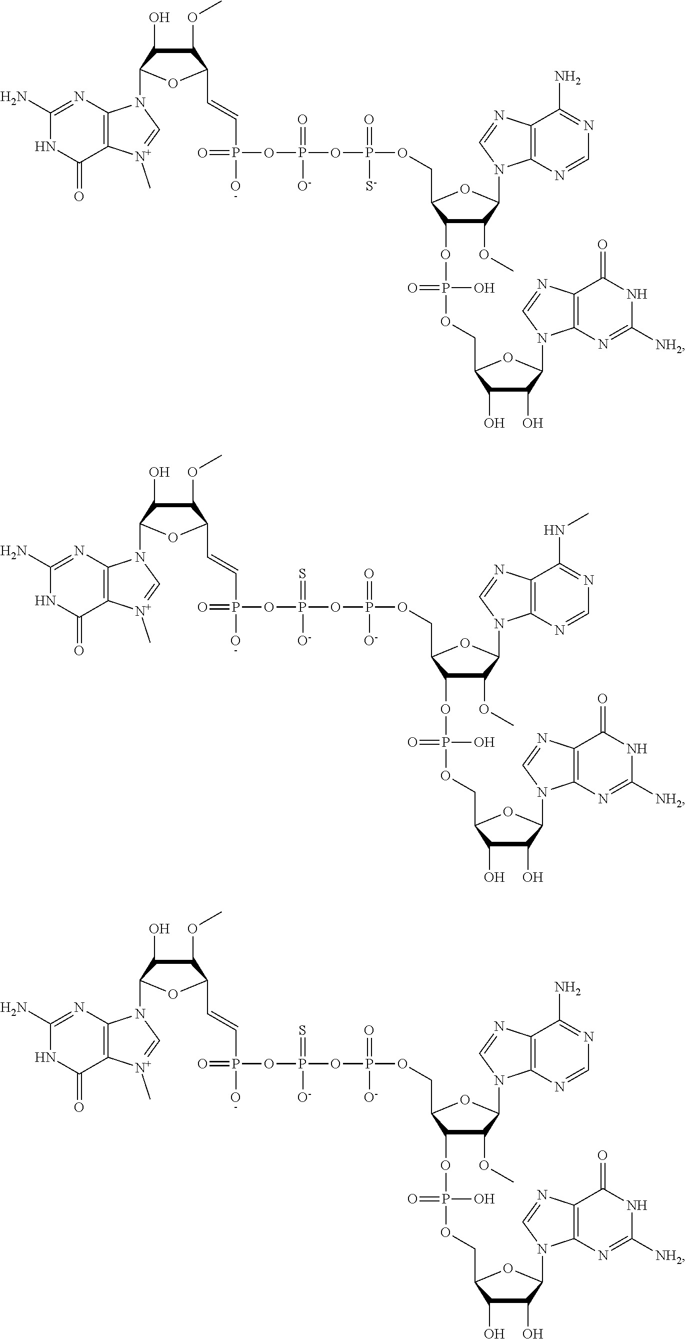

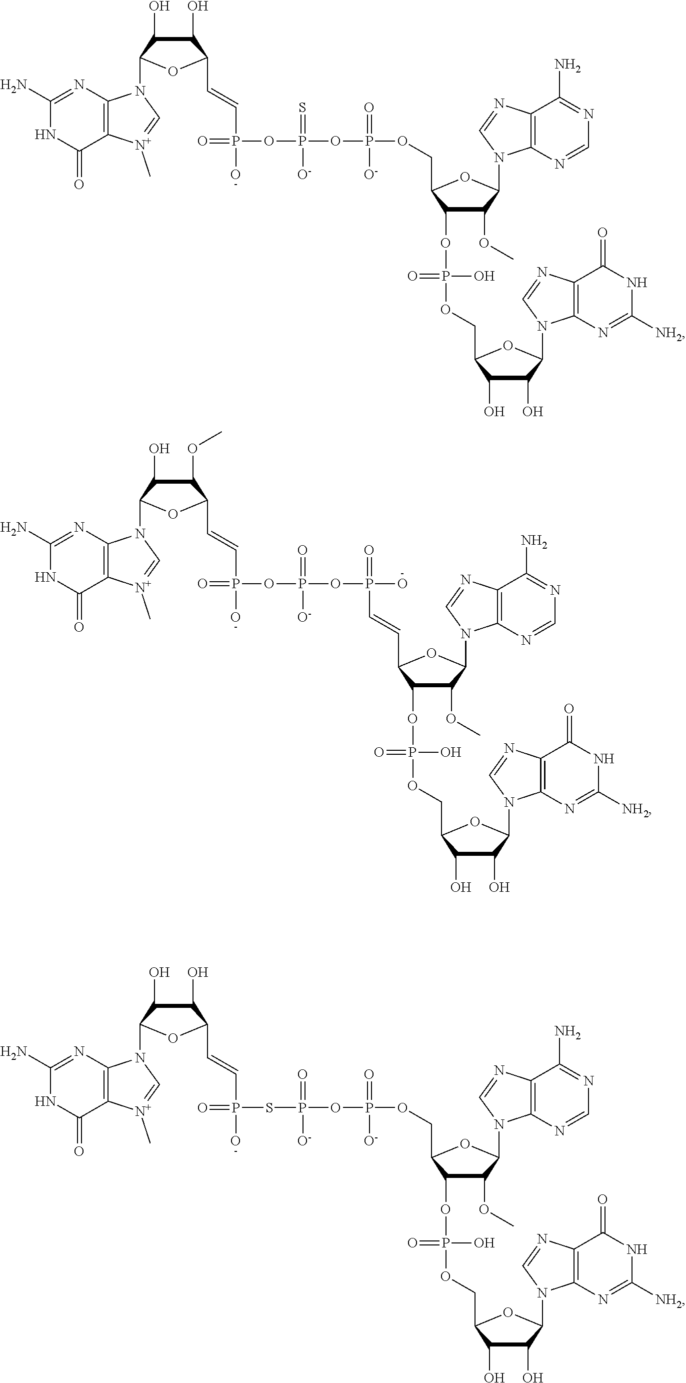

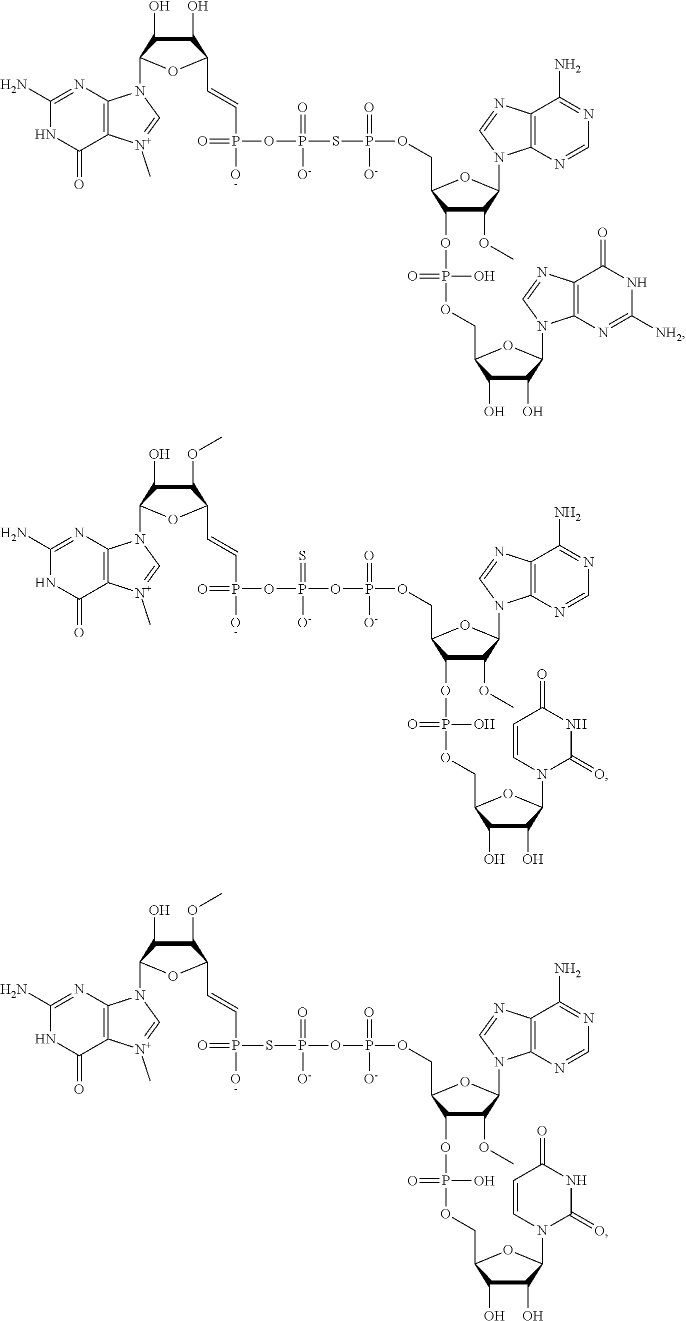

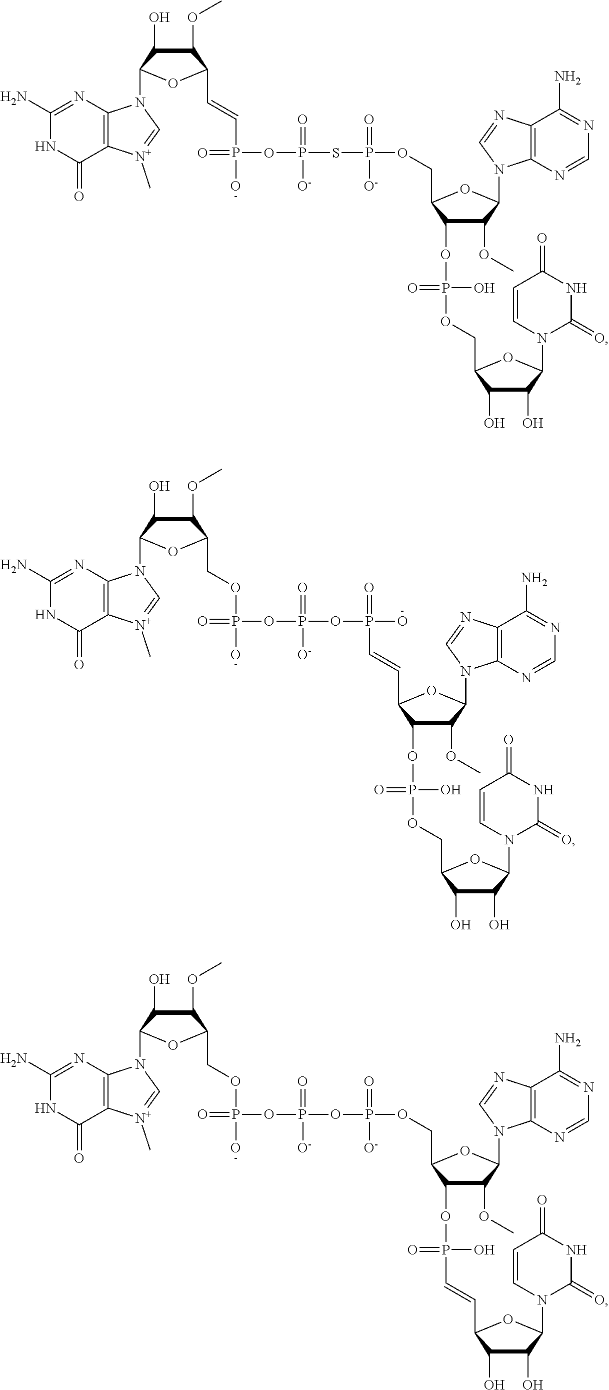

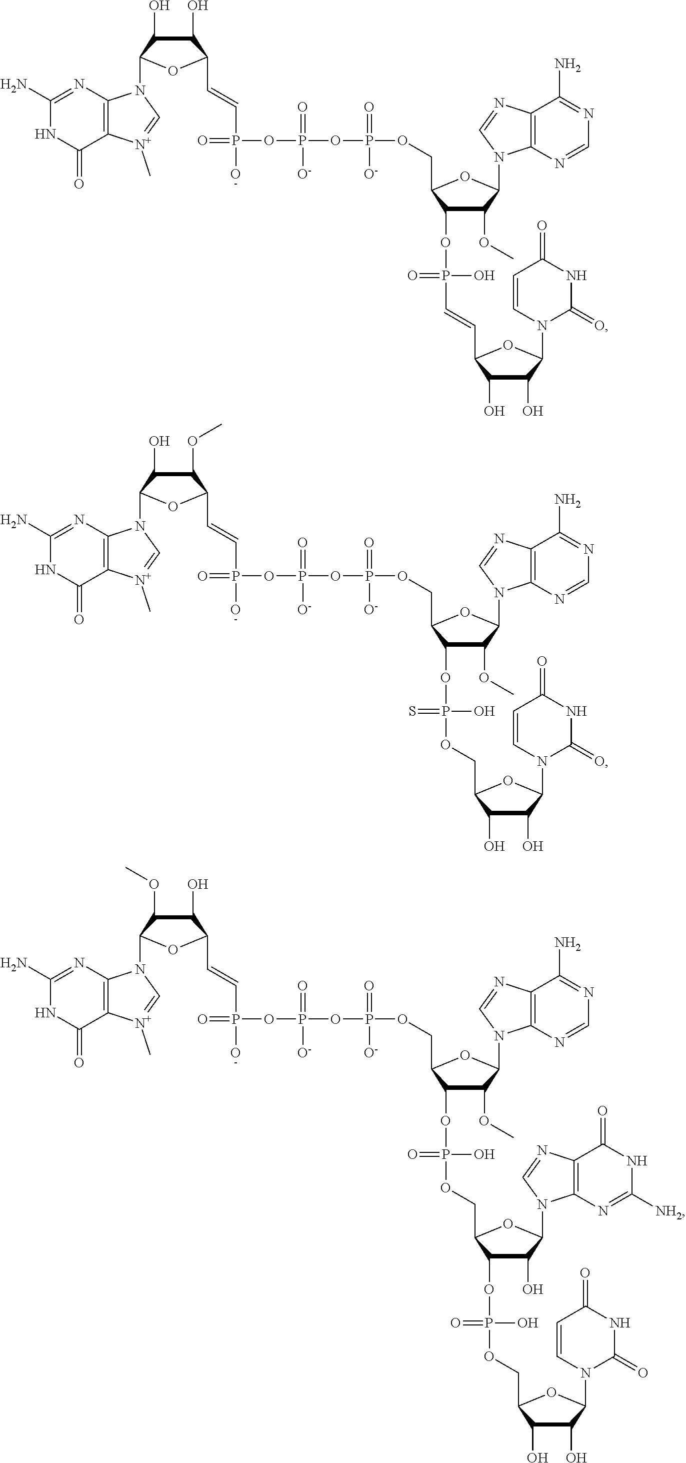

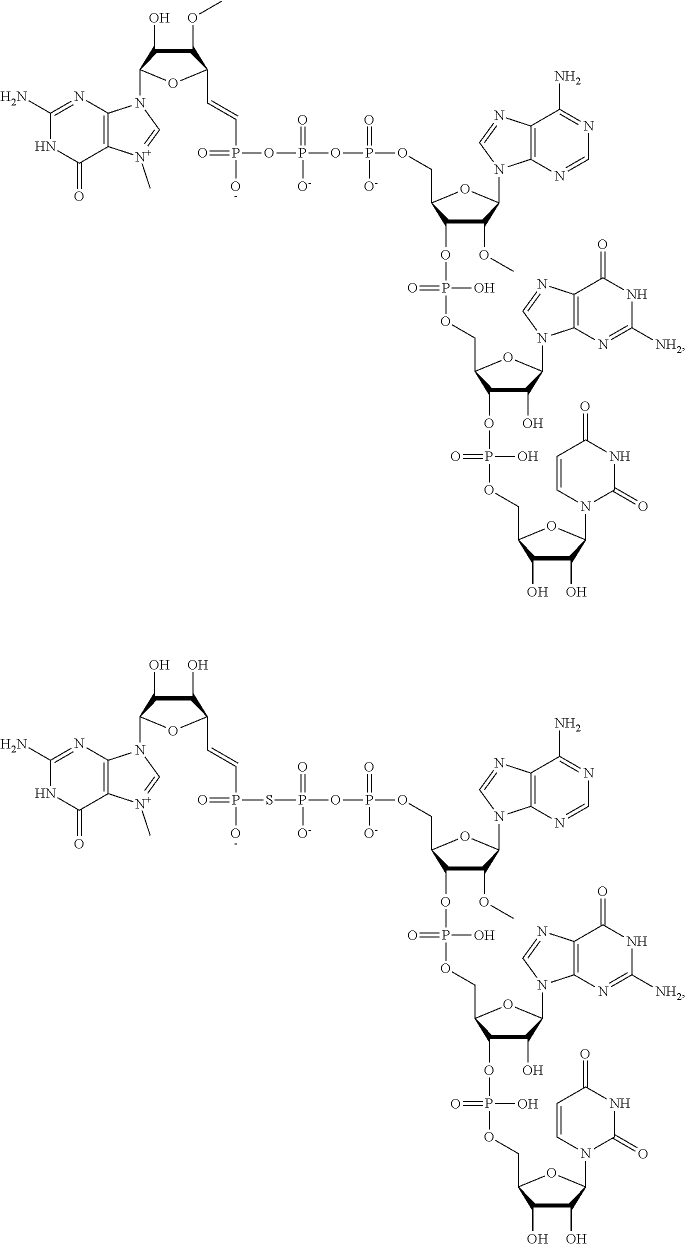

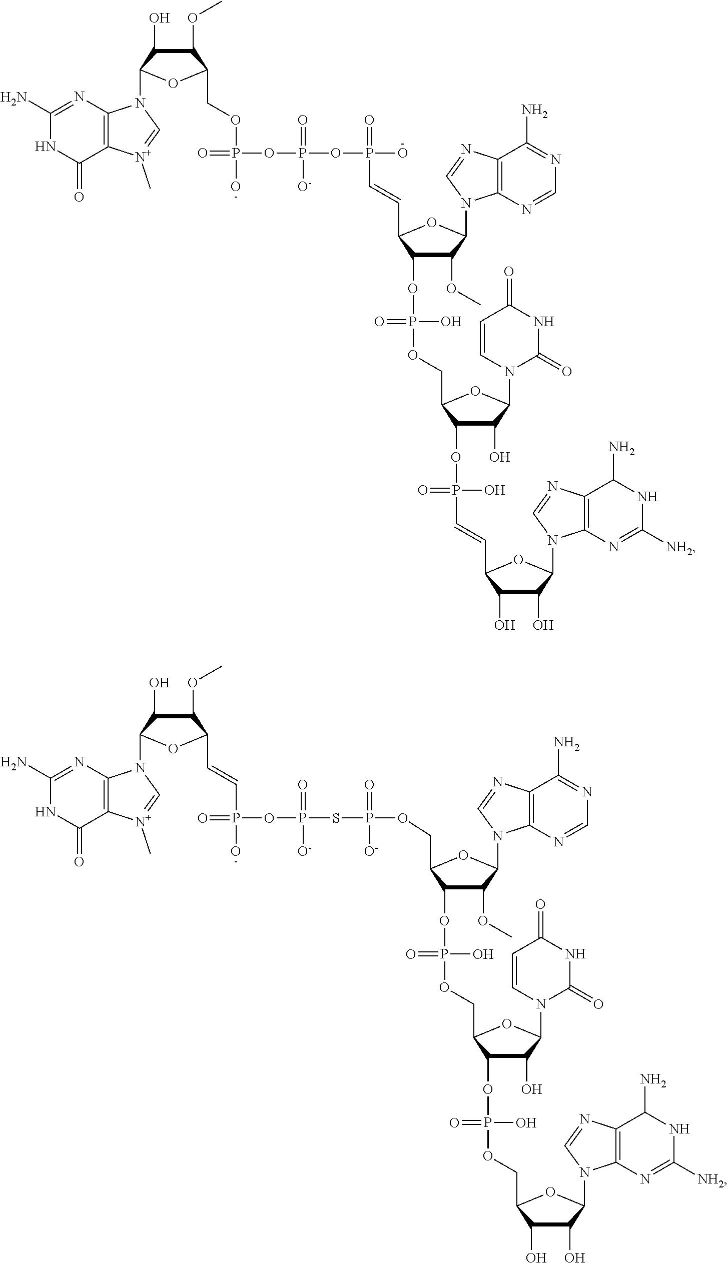

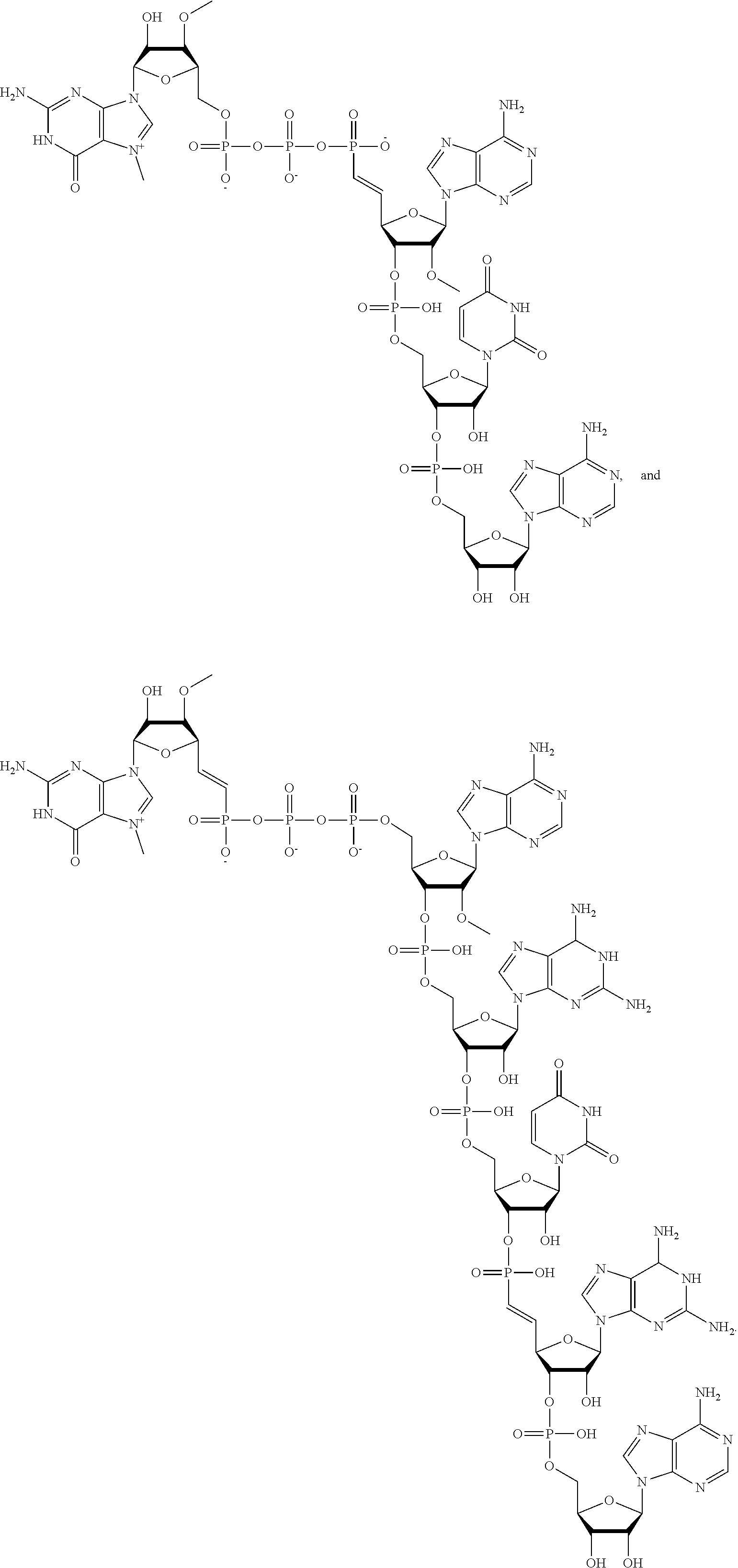

9. The capped polynucleotide according to any one of claims 1-6, wherein the capped polynucleotide is any of following compounds or in a salt form thereof:

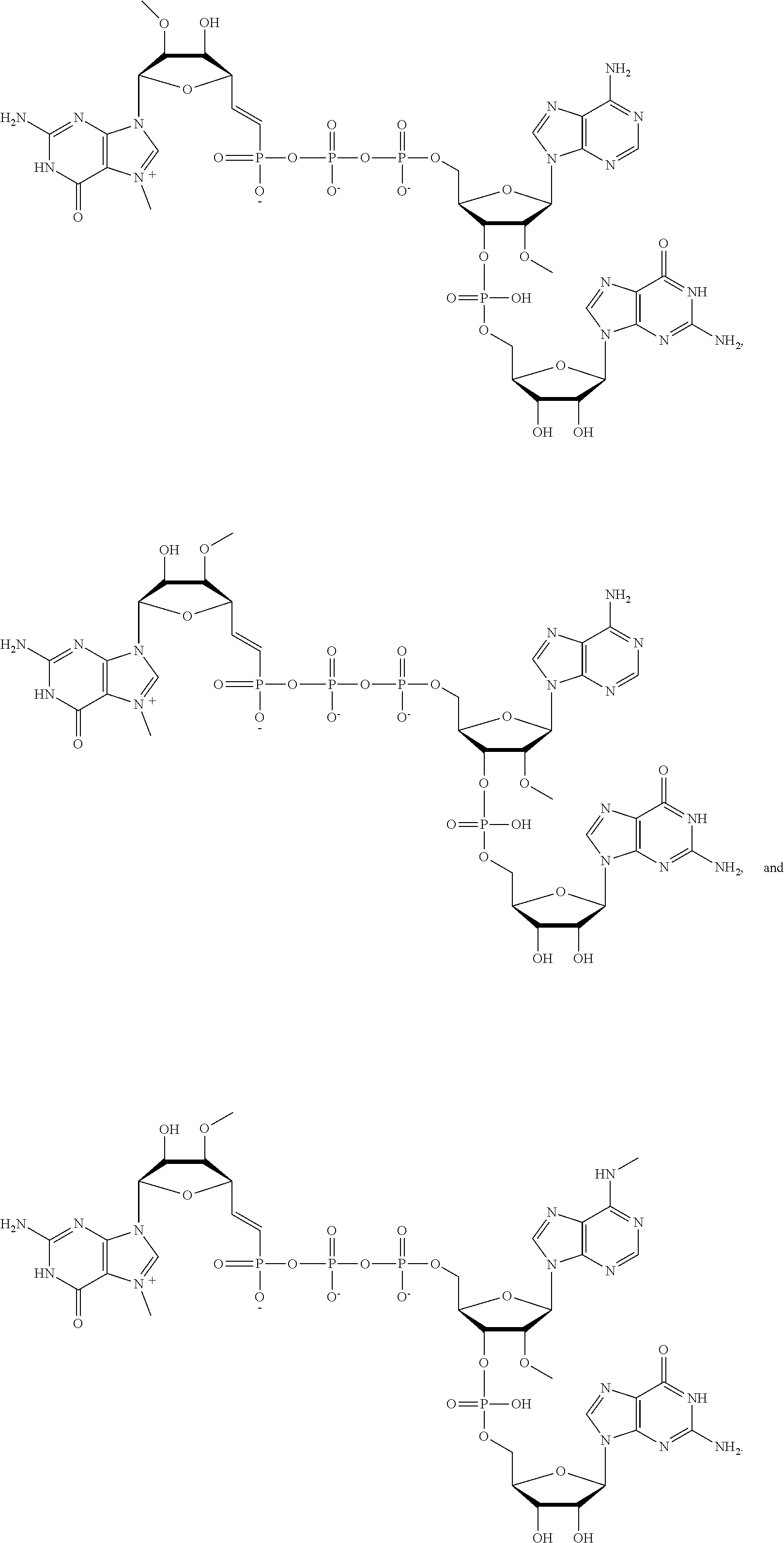

10. The capped polynucleotide according to any one of claims 1-6, wherein the nucleobase comprises any of natural nucleobase or modified nucleobase;
optionally, the natural nucleobase is any one selected from adenine, uracil, guanine, hypoxanthine, cytosine, thymine, adenine derivative, uracil derivative, guanine derivative, cytosine derivative, and thymine derivative, and
optionally, in the modified nucleobase, a modifying group is any one selected from N⁶-methyladenine, N¹-methyladenine, N⁶-2′-O-dimethyladenosine, pseudouridine, N¹-methylpseudouridine, 5-iodouridine, 4-thiouridine, 2-thiouridine, 5-methyluridine, pseudoisocytosine, 5-methoxycytosine, 2-thiocytosine, 5-hydroxycytosine, N¹-methylcytosine, 5-hydroxymethylcytosine, hypoxanthine, N¹-methylguanine, N¹-methylguanine, and isoguanine.

11. A composition for preparing a capped mRNA, wherein components thereof comprise a template DNA and the capped polynucleotide according to any one of claims 1-10, and the template DNA is designed to contain a transcription site complementary to the capped polynucleotide.

12. A preparation method for a capped mRNA, comprising a step of performing transcription using the composition for preparing the capped mRNA in claim 11.

13. A capped mRNA, comprising a main body mRNA fragment and the capped polynucleotide according to any one of claims 1-10, 5′ of the main body mRNA fragment being linked with the capped polynucleotide.

14. A preparation method for pharmaceutical proteins, comprising a step of performing translation with the capped mRNA according to claim 13 as a template.

15. A pharmaceutical protein, prepared by the preparation method for pharmaceutical proteins according to claim 14.

16. Use of the capped mRNA according to claim 13 in preparation of a medicament or a vaccine for treatment of cancer.

17. A pharmaceutical preparation, comprising the capped mRNA according to claim 13, and a vector for carrying the capped mRNA, wherein the vector comprises any of a lipid nanoparticle, a liposome, a polymer nanoparticle, a solid lipid nanoparticle, and an emulsion.
